(19) United States
(12) Patent Application Publication
(10) Pub. No.: US 2011/0236373 A1
(43) Pub. Date: Sep. 29, 2011

(54) Method of detecting antibodies and antibody-hiv virion complexes

(76) Inventors: Georgia Tomaras, Durham, NC (US); Barton F. Haynes, Durham, NC (US); Liu Pinghuang, Durham, NC (US); Nicole Yates, Durham, NC (US); Kwan-Ki Hwang, Durham, NC (US)

(21) Appl. No.: 12/998,211

(22) PCT Filed: Sep. 24, 2009
(86) PCT No.: PCT/US2009/005293

§ 371 Date: Mar. 25, 2011

Related U.S. Application Data
(60) Provisional application No. 61/100,219, filed on Sep. 25, 2008.

(51) Int. Cl. A61K 39/395; C12Q 1/70; C07K 16/42; A61P 31/18

(52) U.S. Cl. 424/131.1; 435/5; 530/387.2

(57) ABSTRACT

The present invention relates, in general, to HIV and, in particular to a method of detecting anti-HIV antibodies and antibody-HIV virion complexes.

This application claims priority from U.S. Provisional Application No. 61/100,219, filed Sep. 25, 2008, the entire content of which is incorporated herein by reference.

This invention was made with government support under Grant No. UO1 AI067854 awarded by the National Institutes of Health. The government has certain rights in the invention.

### TECHNICAL FIELD

The present invention relates, in general, to HIV and, in particular to a method of detecting anti-HIV antibodies and antibody-HIV virion complexes.

### BACKGROUND

The development of a preventive HIV-1 vaccine is a global priority (12). A major roadblock in development of a preventive HIV-1 vaccine is the inability to induce protective antibodies by vaccines or natural infection. Studies in non-human primates have demonstrated that passive infusion of broadly neutralizing anti-HIV-1 monoclonal antibodies prevents infection by simian-human immunodeficiency viruses (SHIVs) (29, 41, 64). Thus, if sufficiently high levels of broadly neutralizing antibodies were present at the time of transmission, protection from HIV-1 infection may be possible. However, to date there is no immunogen formulation that consistently induces broadly neutralizing anti-Env antibodies. Moreover, autologous neutralizing antibody responses do not occur until months after transmission (1, 24, 50, 60). The window of opportunity during which a protective antibody might extinguish HIV-1 after the initial transmission event is uncertain, but is likely to be limited to the period of time prior to establishment of the latent pool of HIV-1 infected CD4+ T cells (34, 61). Although viral latency is certainly established at the time of seroconversion (6), it may be as early as a few days after transmission (18).

An important obstacle to the development of an effective HIV vaccine is the inability to induce antibodies that neutralize primary HIV-1 strains across all genetic subtypes (17, 42). While multiple forms of HIV-1 envelope-based vaccines express epitopes to which rare broadly neutralizing human mAbs bind (i.e. Envs are antigenic), these vaccines have not been immunogenic and have failed to induce broadly neutralizing antibodies against the gp120 CD4 binding site shown to be involved in neutralization breadth (38), the membrane proximal external region (MPER) of gp41 (44, 48), or against gp120 carbohydrate Env antigens (51) in animals or humans.

HIV-1 seroconversion has been reported to occur over a wide range of time, when estimated from the onset of clinical acute HIV-1 infection (AHI) (5, 30, 45); however, the timing of seroconversion of HIV antibodies of particular specificities and isotypes has not been precisely quantified relative to the first time of detectable plasma viremia. Anti-HIV-1 IgM reactive with virus-infected cells has been detected during the course of AHI (10, 11), but the timing of these antibodies and the presence of IgM-virion immune complexes relative to the first detection of viral RNA in AHI have yet to be defined. It is known that autologous neutralizing antibodies only arise months after the first appearance of HIV-specific antibodies (1, 24, 50, 60). Critical questions for understanding the role of early HIV-1 antibodies in the control of HIV-1 are, first, what is the nature and timing of the earliest anti-HIV-1 antibodies and second, what are the contributions of these antibodies in the control of viral replication after transmission?

The present invention results, at least in part, from studies designed to investigate the timing of specific anti-envelope (Env) antibody responses from the eclipse phase (time between transmission and detectable viremia) (19) through 6-12 months of established infection, and model the effect of B cell responses on control of initial plasma viremia. The results demonstrate that the earliest detectable antibodies to HIV-1 are in the form of virion-antibody immune complexes followed 5 days later by free anti-gp41 IgM plasma antibodies. Mathematical modeling of viral dynamics suggests that the initial Env gp41 antibody responses have little effect on control of initial viral replication.

The invention provides methods of detecting anti-HIV antibodies and virion-antibody complexes.

### SUMMARY OF THE INVENTION

The invention relates generally to HIV. More specifically, the invention relates to methods of detecting anti-HIV antibodies and antibody-virion complexes.

Objects and advantages of the present invention will be clear from the description that follows.

### BRIEF DESCRIPTION OF THE DRAWINGS

FIGS. 1A and 1B. (FIG. 1A) Viral load kinetics of 21 HIV+ seroconversion plasma donor panels (eclipse phase clade B infection) were determined. The alignment of the subjects was by T₀, the first day that VL reached 100 copies/ml. (FIG. 1B) Histogram displaying the total number of samples studied for each day, relative to the first detectable day of viremia (T₀). Bins represent intervals of 10 days.

FIGS. 2A and 2B. (FIG. 2A) Kaplan Meier plot of anti-gp41 and anti-gp120 antibody responses in the Eclipse Phase Clade B plasma donor cohort. The solid line shows the increasing percentage of the population that develops HIV specific antibody responses each time interval following the calculated T₀. The dashed lines indicate the upper and lower point-wise confidence intervals respectively. (FIG. 2B) Pairwise comparison of the timing of anti-Env antibody responses compared anti-Gag (p24, p17, p55) and anti-Pol (p31) responses in the Eclipse Phase Clade B plasma donor cohort. The solid line (from left to right) indicates the median day of antibody elevation from T₀ and the gaps in the line indicate the HIV specific antibody responses that group together relative to their time of elevation from T₀. The median time for appearance of IgG anti-gp41 antibody was 13.5 days (FIG. 2A, left panel), while the median time for appearance of IgG gp120 antibody was 28 days (FIG. 2A, right panel).

FIGS. 3A-3C. Anti-gp41 IgM antibodies are the first detectable HIV antibodies and autologous gp140 transmitted Env or consensus Env gp140 proteins are equally sensitive for the detection of the first antibody isotypes in HIV infection. (FIG. 3A) IgM antibodies (FIG. 3B) IgG antibodies (FIG. 3C) IgA antibodies were detected using either consensus gp140 (ConB) or autologous Env (6246 Env). The asterisk indicates the plasma sample from which the autologous gp140 Env was derived. Consensus gp160 oligomer detects anti-gp41 antibodies at the same time as autologous gp140 Env oligomers.

FIGS. 4A-4D. Kinetics of anti-gp41 specific antibody isotypes in acute HIV infection. Representative examples of (FIG. 4A) sequential development and (FIG. 4B) simultaneous development of early HIV specific antibody responses are shown. (FIG. 4C) The percentage of patients in each of the three cohorts that displayed different kinetic patterns. (FIG. 4D) Simultaneous Development of Gag Specific Antibody Responses. Anti-p55 antibodies of the IgM, IgG and IgA isotypes were measured for all subjects in the Eclipse Phase Clade B Cohort. Pt. 12007 could not be aligned to T₀ due to the large interval between the first RNA positive sample and the last RNA negative sample. However, the short interval between antibody positive and antibody negative enabled measurement of antibody isotype kinetics, so the panel was aligned to T₀ as the first RNA positive sample.

FIG. 5. HIV immune complexes produced at a median time of 8.0 days post T₀. The detection of immune complexes for Pts. 9015, 12008, 9077, 9079, 9021 and 9076 are aligned to T₀ and plotted in comparison to the detection of free antibody responses.

FIGS. 6A and 6B. Ontogeny of complement binding antibodies during acute HIV-1 infection in times post T₀. Two representative patients from the eclipse phase cohort (6240 (FIG. 6A), 6246 (FIG. 6B)) that had detectable HIV specific antibodies were assessed for complement activation with an early virus isolate, HIV QH0692, and a lab adapted isolate, HIV SF162.

FIGS. 7A and 7B. (FIG. 7A) No hypergammaglobulinemia observed within the first 40 days of acute infection. Total antibody levels were measured at the first HIV(−) sample and the last sample in the panel (HIV+). The median concentration across panels is indicated. (FIG. 7B) Detection of rheumatoid factor (RF) during HIV acute phase viremia. IgM rheumatoid factor was measured using standard ELISA detection with positive rheumatoid factor controls. VL=viral load in RNA copies/ml.

FIG. 8. Modeling the effect of antibody on plasma viremia in AHI with the target cell limited model. The target cell limited model is the best-fitting model for the plasma donors studied except 9032. For 9032, a model with virion clearance enhanced by the sum of anti-gp41 IgM and IgG provides the best fit.

FIGS. 9A and 9B. (FIG. 9A). Viral load kinetics of 14 subjects from the Trinidad and Tobago Cohort and 10 CHAVI 001 Cohort that were utilized to characterize HIV-1 specific antibodies. The alignment of the subjects was by the first day of enrollment in the study. (FIG. 9B). The distribution of samples relative to first day of enrollment.

FIGS. 10A-10C. Autologous gp140 transmitted Env from Subject 6240 or consensus Env gp140 proteins are equally sensitive for the detection of the first antibody isotypes in HIV infection. (FIG. 10A). IgM antibodies (FIG. 10B) IgG antibodies (FIG. 10C) IgA antibodies. The asterisk indicates the plasma sample from which the autologous gp140 Env was amplified, sequenced and cloned for the expression of gp140 oligomers.

FIG. 11. Development of non-neutralizing cluster II anti-MPER antibodies (cII-MPER), Cd4i and CD4bs antibodies in 14 CAPRISA and 12 Trinidad patients from enrollment in the acute infection study.

FIGS. 12A and 12B. C14 mAb captures virions to form immune complexes.

FIG. 13. Mucosal HIV-1 gp41 Env IgM present early in acute infection—Pt. 700-01-047-0.

FIG. 14. Mucosal HIV-1 gp41 Env IgA specific for the 2F5 epitope present early in acute infection—Pt. 700-01-047-0.

FIGS. 15A-15K. (FIGS. 15A-15F) Characterization of C14-2 IgM derived from uninfected terminal ileum sample (C14). (FIGS. 15G-15K) Characterization of F3 IgM derived from terminal ileum of a patient with acute HIV-1 infection.

### DETAILED DESCRIPTION OF THE INVENTION

The present invention relates to methods of detecting specific antibodies and antibody-HIV virion complexes in the sera or plasma of HIV-1 exposed and/or infected individuals. It has previously been shown that HIV-1 transmission results in the production of antibodies that bind specific HIV antigens and commercially available antibody/antigen tests are available that test for antibody positivity to HIV. This invention provides a method for detecting antibody-virion complexes before free antibody is detected by currently available commercial tests. This invention makes possible assay development that can stage individuals in acute infection so that the time since infection can be approximated.

In the study described in the Example below, it is shown that the initial B cell response to the transmitted/founder virus is to HIV-1 gp41 Env, with responses to gp120 delayed by an additional 14 days. Mathematical modeling of the effects of initial IgM and IgG gp41 antibodies on viral kinetics in the plasma donor cohort revealed little if any effect of the initial antibody response on control of acute phase plasma viremia.

The timing and specificity of the initial antibody response to HIV-1 Env is of interest for several reasons. First, the window of opportunity for a vaccine to extinguish the transmitted or founder strain of HIV-1 is likely quite short, and the timing of this window will vary from subject to subject depending on the time of establishment of the latent pool of CD4+ T cells. That post-exposure prophylaxis does not protect beyond 24 hours after SIV challenge in rhesus macaques (18) implies that the window of opportunity may be 10 days or less in humans (61). Moreover, early appearance of evidence of systemic inflammation and acute phase reactants in plasma at 5 to 7 days before T₀ (Kessler, B, McMichael, A, and Borrow, P, personal communication), as well as the appearance of plasma analytes of apoptosis 7 days after T₀ (22), add support to this short estimated window of opportunity for vaccine efficacy. Given that a narrow window of time might exist for antibodies to be protective and given that immune complexes only arise ˜18 days after transmission (8 days after T₀ with an estimate of time from transmission to T₀ of a mean of 10 days, range 7-21 (7, 11, 14, 21, 40, 52)), then the first antibody response to HIV-1 is quite delayed relative to when it optimally should occur to either extinguish or control transmitted/founder HIV-1 strains.

This study is the first demonstration of virion-antibody complexes during the initial phase of viremia in acute HIV-1 infection. Previous work, looking at later times in acute infection, did not find immune complexes early in HIV-1 infection, but rather found immune complexes only in chronic infection (16). The presence of these early immune complexes during acute infection raises the question of whether antibody-coated virus remains infectious; work is ongoing to determine the infectivity of opsonized virus (Montefiori, D. C., Tomaras, G. D., Haynes, B. F., unpublished). However, it is well established that the likelihood of HIV-1 transmission is highest during acute infection. Taken together, these data suggest an HIV-1 evasion strategy wherein the transmitted/founder virus initially induces antibodies that bind virions yet are non-neutralizing. Further work is needed to decipher the specificities and avidities of the antibodies in the immune complexes present at 8 days post T₀ to determine if these initial antibody responses could be protective if a vaccine were able to prime for an early boost of the timing and magnitude of these antibodies after HIV-1 transmission.

That the initial B cell response to Env selectively recognized gp41 also was of interest. Li et al. recently demonstrated that when broadly neutralizing antibodies do appear, they appear late and include antibodies against the CD4 binding site on gp120 (38). While there are broadly neutralizing epitopes on gp41 in the MPER, like broadly neutralizing CD4 binding site antibodies, neutralizing antibodies to the MPER are rarely made, and when they are made, require>2 years after transmission to arise (Shen., X, Tomaras, G. D., unpublished observations). Thus, the host-pathogen interactions occurring during and immediately after transmission results in a delay in recognition of gp120 by host B cells until after the latent pool of infected cells is likely established.

Polyclonal activation of B cells occurs in chronic HIV-1 infection and as well has been reported in early HIV-1 infection (47). No polyclonal hypergammaglobulinemia was found in plasma donors, but plasma rheumatoid factor was found in ˜30% of subjects. Thus, polyclonal B cell activation does occur early on as signaled by the detection of this autoantibody, likely indicating triggering of CD5+ B cells that are producers of rheumatoid factor autoantibodies (26).

Also of interest is that there was heterogeneity in the pattern of Ig class-switching seen following HIV-1 transmission. It has been shown that the simultaneous appearance of anti-HIV IgM, IgG, and IgA are unlikely to be due to the presence of immune complexes that mask detection of antibodies, because immune complexes appeared at the same time as free antibody in half of the subjects. Other potential explanations for simultaneous appearance of IgM, IgG, and IgA anti-HIV-1 antibodies in plasma include: prior exposure to HIV-1, and primary T cell independent B1 and marginal zone B cell responses to HIV-1 following transmission (9).

If the simultaneous appearance of IgM, IgG and IgA to Env and Gag represent prior exposure of ˜60% of subjects to HIV-1 antigens and represents a rapid secondary response to HIV-1 full infection, then an atypical aspect of the response is that the putative “secondary” response occurs at exactly the same time as the primary IgM response (day 13.0 after T₀) occurs in those with sequential appearance of anti-Env and Gag IgM, IgG and IgA. If the simultaneous response is indeed secondary from prior HIV-1 exposure, then it should occur approximately 7 days earlier than observed. Thus, it is unlikely that simultaneous appearance of IgM and class-switched antibodies in plasma in over ⅔ of AHI studied indicate prior exposure to HIV-1.

Soon after transmission in both humans and non-human primates when infection is established, there is a severe depletion of CD4 T lymphocytes (4, 57) that could lead to a lack of sufficient CD4 help for stimulation of B cell responses. The early depletion of CD4+CCR5+ T lymphocytes with massive apoptosis could in addition to altering T cell homeostasis lead as well to suppression of an initial protective B cell response (22, 43). Thus, elicitation of initial T independent antibody responses, in the setting of T cell depletion, could be responsible for simultaneous appearance of IgM, IgG and IgA anti-HIV-1 antibodies. A similar T independent pattern with simultaneous appearance of IgM, IgG and IgA anti-pneumococcal antibody occurs following pneumococcal vaccine (9).

It was important to model both the antibody timing and the viral load dynamics to determine any salutary or detrimental effects of early antibody responses on control of plasma viremia. As a null model, a simple target-cell limited model was used that includes no effect of humoral or cellular immune responses (53). For 5 of the 6 plasma donors for which VL data was available that extended past the viral load peak and out to day 40 past T₀ this model gave good agreement with the VL data. Nonetheless, the question was asked as to whether the fit could be improved by using a model that incorporated any of a variety of known functional effects of antibody. Not surprisingly, it was only for the one plasma donor, 9032, for which the target cell limited model did not give good agreement with the VL data that an improvement was seen when including antibody in the model. Interestingly, this donor was unusual in that the peak VL was significantly lower than in the other donors (3.4×104 copies/ml). Taken together, these analyses suggested that for most donors early antibody had little functional consequence for the control of viremia.

If early antibody induced by the transmitted virus had any antiviral effects, then antibody-induced viral escape should be detected after appearance of complexed or free antibody in plasma. In this regard, Keele et al. (35) have recently sequenced the transmitted founder virus for the plasma donors studied in this report and found that virus sequences at 14 days after T₀ conformed to a model of random viral evolution thus showing no evidence of early antibody induced selection.

For HIV-1, functional consequences of antibody binding could include virus neutralization on T lymphocytes or macrophages (31, 32), antibody-dependent cellular cytotoxicity (ADCVI/ADCC), complement-mediated neutralization, antibody Fc-mediated effector functions, virolysis and/or inhibition of transcytosis. A recent study (29) suggested that the concentrations of antibodies mediating the different anti-viral functions may be an important consideration for complete virus elimination, since Fcγ-receptor-binding function requires higher antibody concentrations than are required for virus neutralization. In addition, antibody and complement-mediated virion lysis can develop in acute infection and can correlate with plasma virus load during the acute stage of infection (33). This antiviral activity did not correlate with neutralizing antibodies and is thought to be an antiviral component of non-neutralizing antibodies. Dendritic cells (DCs) are positioned in mucosae where they are thought to be one of the first cell types to help establish infection (reviewed in (62)). It will be important to determine whether the very earliest antibodies elicited in acute HIV-1 infection can block HIV-1 infection in dendritic cells at mucosal surfaces.

It is clear that the initial B cell response to the transmitted/founder virus does not control initial virus levels during the first 40 days of infection. However, it cannot be ruled out that some antibody specificities elicited after virus transmission may affect a subset of virions but are not substantial enough to significantly affect plasma viremia at the time they appear. A critical question is whether these types of non-neutralizing antibodies could be protective, if present before infection or rather, if a completely different type of inhibitory antibodies will need to be induced by future HIV-1 vaccines. Autologous neutralizing antibodies target envelope variable loops (46, 49, 50, 60), that can arise long after any window of opportunity to extinguish HIV-1 has passed. Thus, an effective HIV-1 vaccine will need to induce antibodies prior to infection that bind native virion envelope molecules and as well will lead to maturation of a rapid secondary neutralizing antibody response within the first week after transmission.

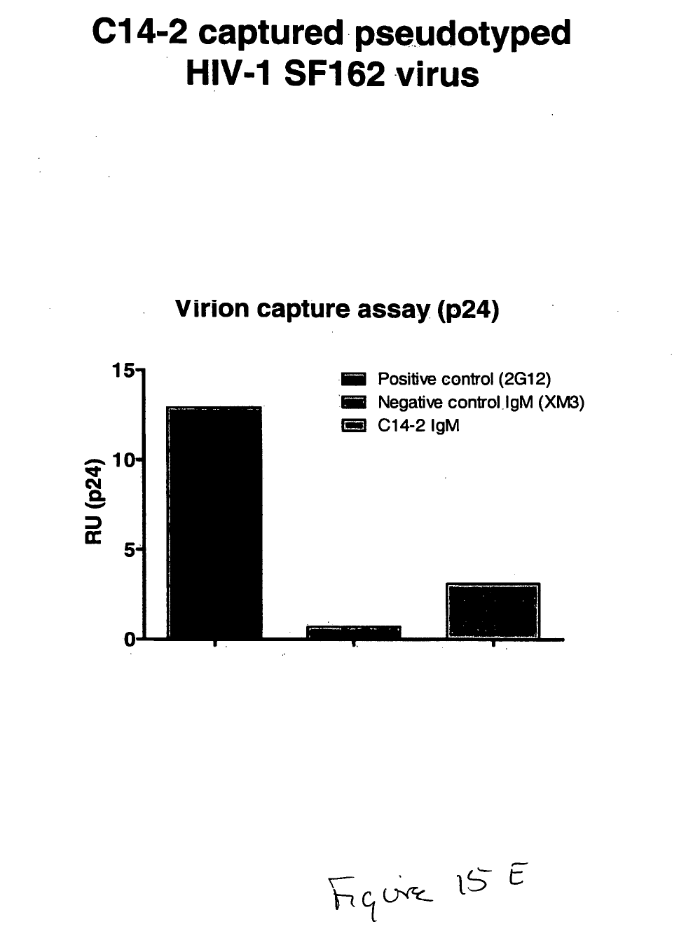

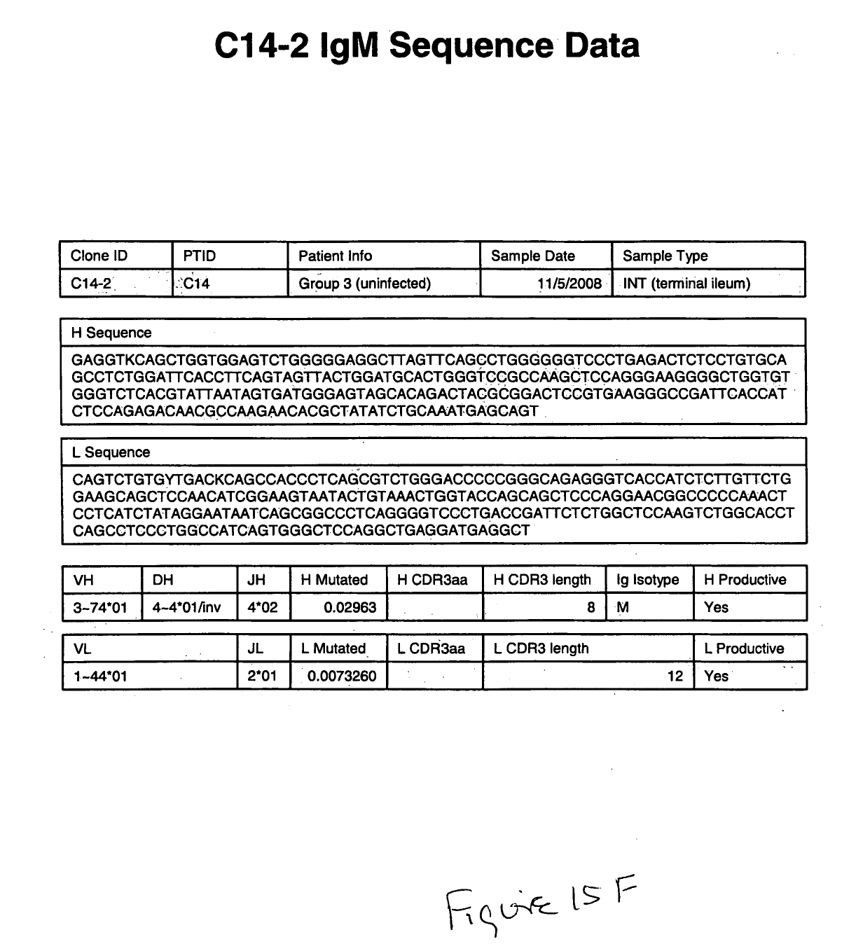

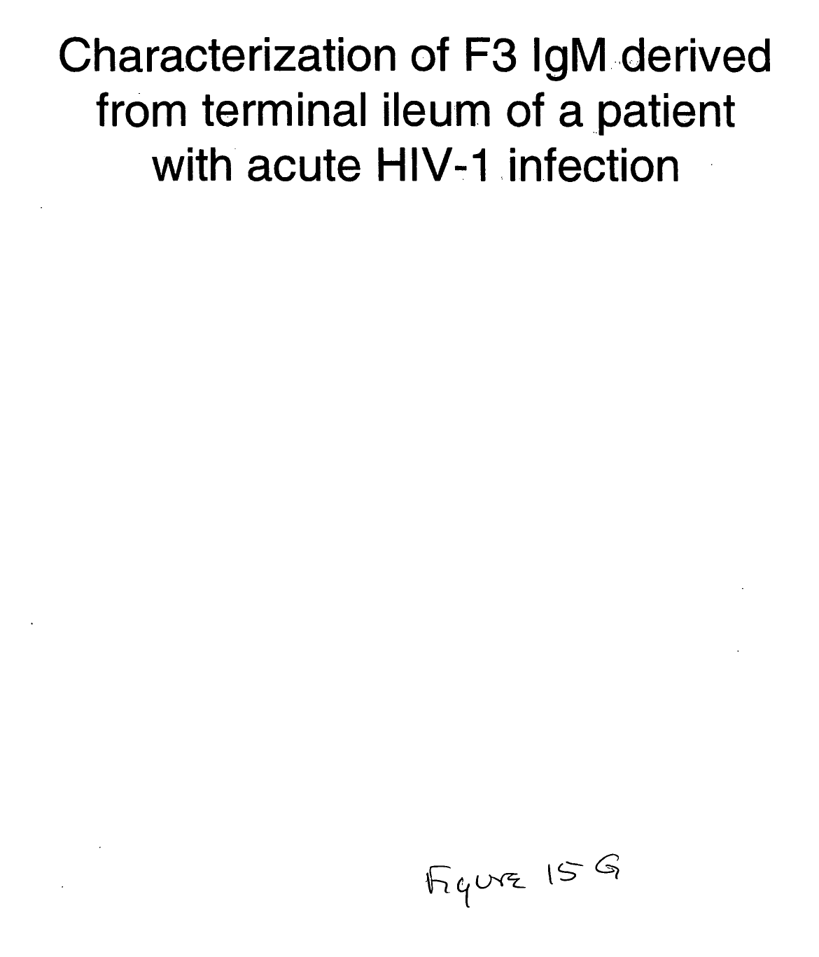

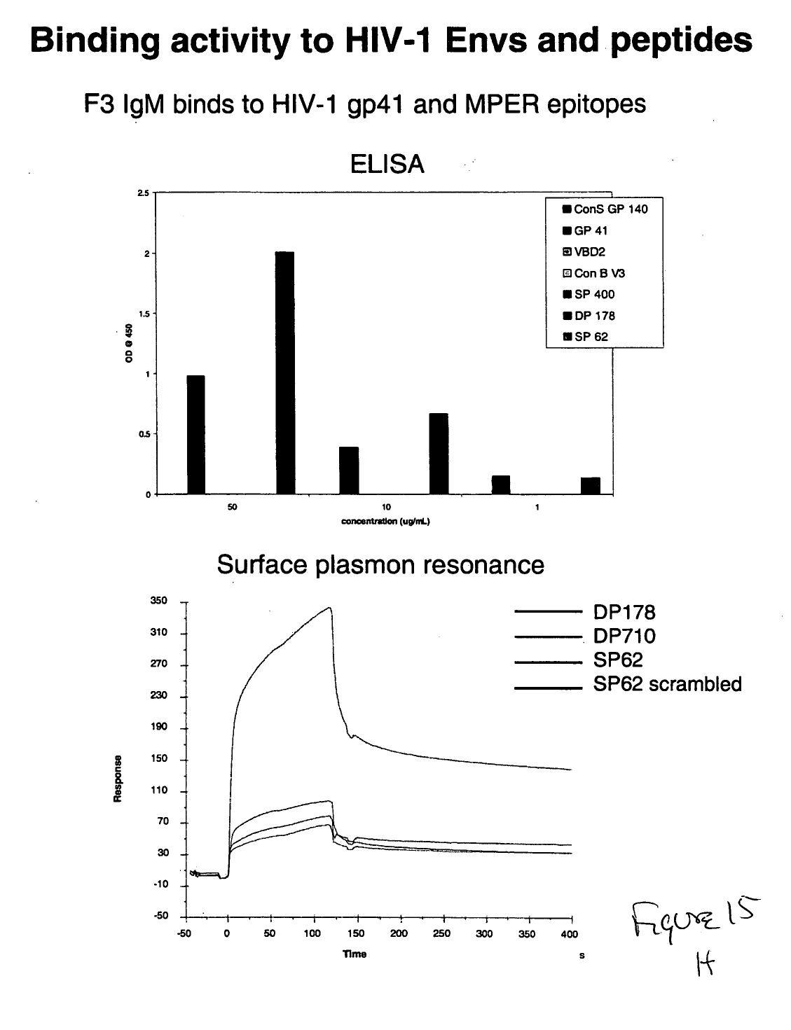

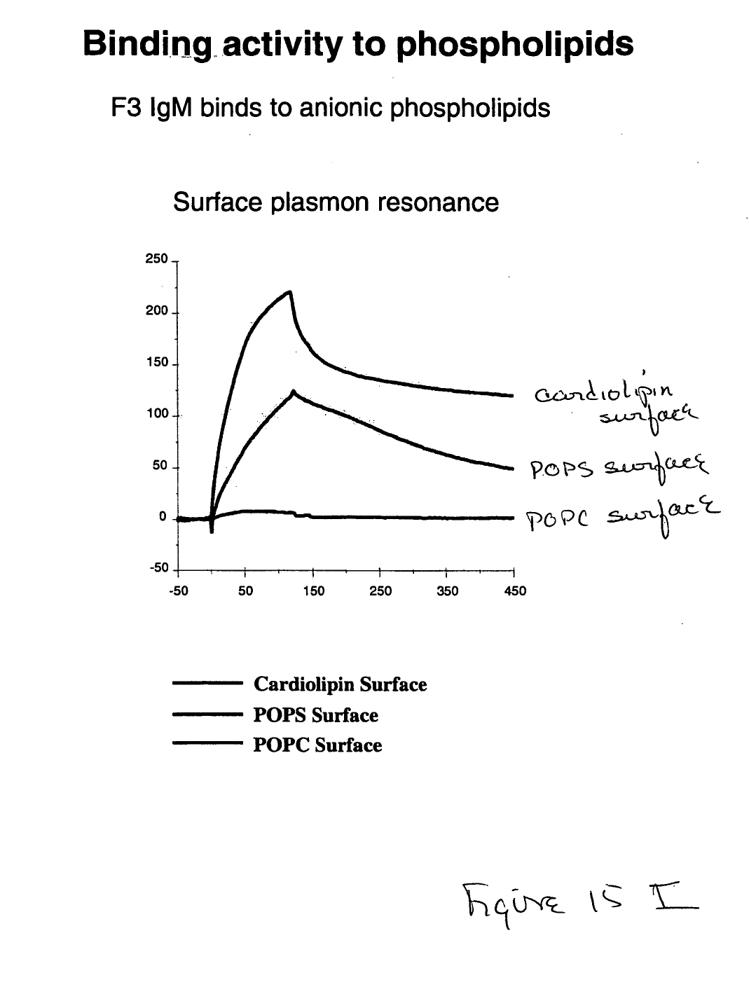

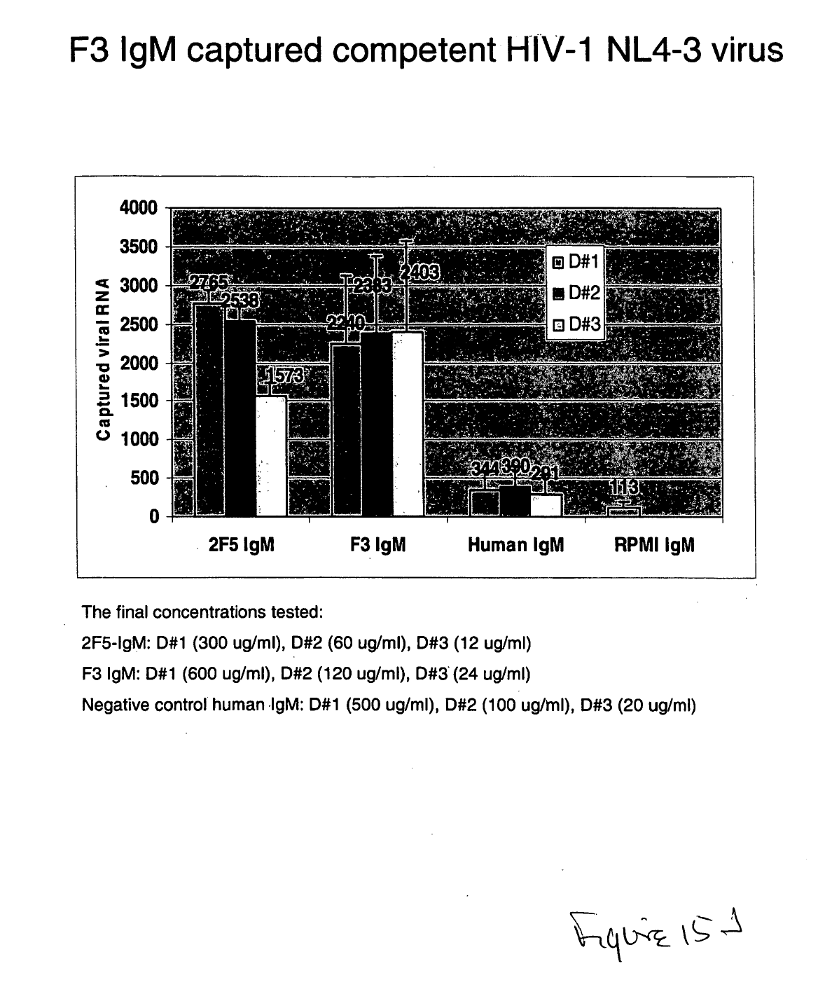

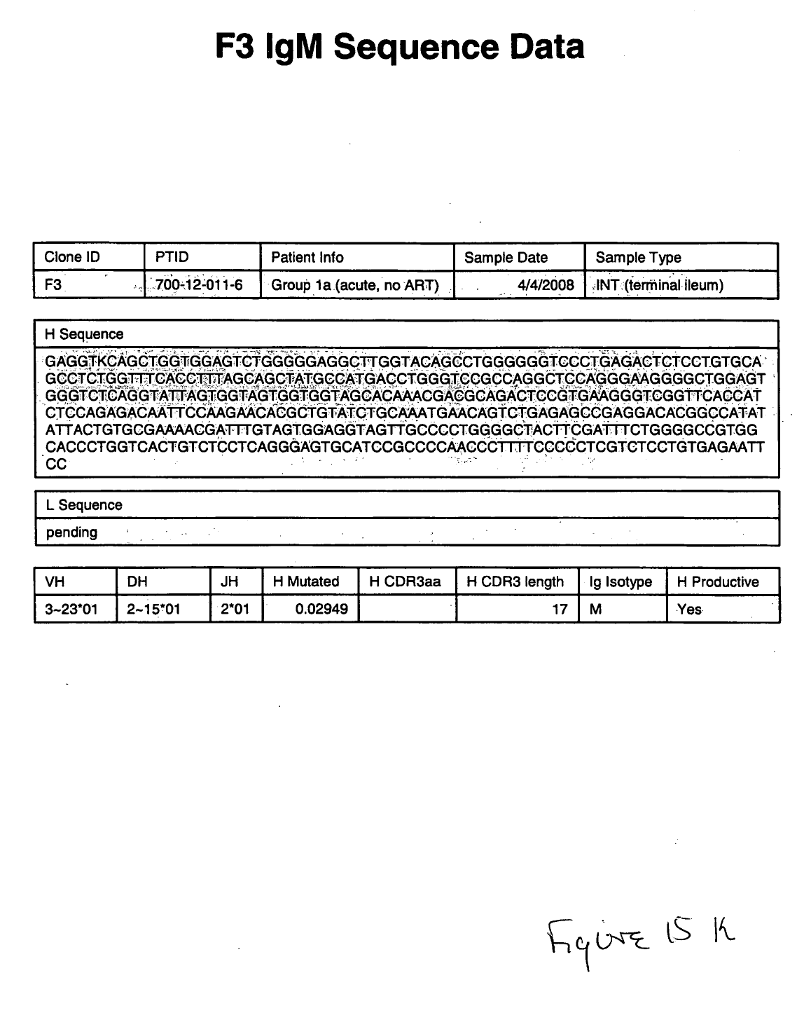

The example of an antibody that binds antibody-HIV virion complexes is a mAb having the variable heavy and variable light sequences of the C14-2 antibody as set forth in FIG. 15F. A further example is an antibody having a heavy chain sequence of the F3 antibody as set forth in FIG. 15K. The invention includes the intact antibody or fragments (e.g., antigen binding fragment) thereof. Exemplary functional fragments (regions) include scFv, Fv, Fab′, Fab and F(ab′)₂ fragments. Single chain antibodies can also be used. Techniques for preparing suitable fragments and single chain antibodies are well known in the art. (See, for example, U.S. Pat. Nos. 5,855,866; 5,877,289; 5,965,132; 6,093,399; 6,261,535; 6,004,555; 7,417,125 and 7,078,491 and WO 98/45331.) The invention also includes variants of the antibodies (and fragments) disclosed herein, including variants that retain the binding properties of the antibodies (and fragments) specifically disclosed, and methods of using same.

The antibodies, and fragments thereof, described above can be formulated as a composition (e.g., a pharmaceutical composition). Suitable compositions can comprise the antibody (or antibody fragment) dissolved or dispersed in a pharmaceutically acceptable carrier (e.g., an aqueous medium). The compositions can be sterile and can be in an injectable form. The antibodies (and fragments thereof) can also be formulated as a composition appropriate for topical administration to the skin or mucosa. Such compositions can take the form of liquids, ointments, creams, gels, pastes and aerosols. Standard formulation techniques can be used in preparing suitable compositions. The antibodies can be formulated so as to be administered as a post-coital douche or with a condom.

The antibodies and antibody fragments of the invention can be used to inhibit or treat HIV infection in a subject (e.g. a human). Suitable dose ranges can depend, for example, on the antibody and on the nature of the formulation and route of administration. Optimum doses can be determined by one skilled in the art without undue experimentation. Doses of antibodies in the range of 10 ng to 20 μg/ml can be suitable.

Certain aspects of the invention can be described in greater detail in the non-limiting Examples that follows. (See also Tomaras et al, J. Virol. 82:12449 (2008)).

### EXAMPLE 1

### Experimental Details

### Subjects Studied

A subset of subjects from four different acute infection cohorts were examined: 21 plasma donors, 12 AHI subjects from the Trinidad cohort (clade B), 14 AHI subjects from the CAPRISA (clade C) cohort and 10 AHI subjects from the CHAVI 001 acute infection cohort.

### Viral Load Testing

Plasma viral RNA was measured by Quest Diagnostics (Lyndhurst, N.J.) (HIV-1 RNA PCR Ultra).

Antigens used in Antibody Binding Assays

The antigens used for direct antibody binding assays are: group M consensus Env CON-S gp140, consensus B gp140, clade B wildtype Env gp120s (produced by either recombinant vaccinia (39) or 293T transfection. IIIB, JRFL, 89.6, as well as the following peptides (Primm Biotech Inc, Cambridge, Mass.) and their sequences. SP400 (gp41 immunodominant region, RVLAVERYLRDQQLLGIWGCSGKLICTTAVPW NASWSNKSLNKI), SP62, gp41 MPER, (QQEKNEQELLELDKWASLWN), 4E10 P, (SLWNWFNITNWLWYIK), Consensus B V3 gp120 region, (TRPNNNTRKSIHIGPG RAFYTTGEIIGDIRQAH), Consensus M V3 CON-S gp120 region, TRPNNN TRKSIRIGPGQAFYATGDIIGDIRQAH. Acute HIV-1 envelope gene sequences were derived from of 4 subtype B acute HIV-1 infected individuals (Subjects 6246, 6240, and 9021 by single genome amplification (SGA) (35). To produce recombinant soluble gp140 proteins, a gp140 env gene construct named gp140C was designed, in which the transmembrane and cytoplasmic domains of HIV-1 Env were deleted and 2 critical Arg residues in the gp120-gp41 cleavage site were replaced with 2 Glu residues. All four gp140C env genes were codon-optimized by employing the codon usage of highly expressed human housekeeping genes, de novo synthesized (Blue Heron Biotechnology, Bothell, Wash.) and cloned into pcDNA3.1/Hygro expression plasmid (Invitrogen, Carlsbad, Calif.) using the standard molecular technology. Recombinant HIV-1 gp140C Env proteins were produced in 293T cells by transient transfection with the resulting plasmids and purified by Galanthus nivalis lectin-agarose (Vector Labs, Burlingame, Calif.) column chromatography (39). Autologous V3 peptides were made from these same Envs.

### Antibody Assay Criteria.

The positivity criterion per antigen per antibody isotype was determined by screening ≧30 seronegatives. A standardized HIV+ positive control is titrated on each assay (tracked with a Levy-Jennings plot with acceptance of titer only within 3 STDEV of the mean) and the average O.D. is plotted as a function of serum dilution to determine antibody titer using a four-parameter logistic equation (SoftMaxPro, Molecular Devices). The coefficient of variation (CV) per sample is ≦15%. Two negative sera and two HIV+ control sera are included in each assay to ensure specificity and for maintaining consistency and reproducibility of between assays. The integrity of raw data acquisition, data analyses are electronically tracked (21CFR part11 compliant).

### Direct ELISAs

Direct ELISAs were performed using consensus clade B or envelope glycoproteins, gp41 proteins, consensus V3 peptides, gp41 immunodominant, and MPER epitopes, as well as autologous V3 and gp140 Env oligomers. ELISAs were conducted in 96 well ELISA plates (Costar #3369) coated with 0.2 μg/well antigen in 0.1M sodium bicarbonate and blocked with assay diluent (PBS containing 4% (w/v) whey protein/15% Normal Goat Serum/0.5% Tween20/0.05% Sodium Azide). Sera were incubated for 1 hour in two fold serial dilutions beginning at 1:25 followed by washing with PBS/0.1% Tween-20. 100 μl Alkaline phosphatase conjugated goat anti-human secondary antibody (Sigma A9544) was incubated at 1:4000 for 1 hour, washed and detected with 100 μl substrate (CBC buffer+2 mM MgCl2+1 mg/ml p-npp [4-Nitrophenyl phosphate di(2-amino-2-ethyl-1,3-propanediol) salt]). Plates were read at 405 nm, 45 minutes.

### Competitive Inhibition Studies (Antibody Blocking Assays)

Competitive inhibition studies (Antibody blocking assays) were performed with 1b12 (CD4BS), 2G12 (anti-CHO), and the MPER mAbs, 2F5 and 3H11. 96 well ELISA plates (Costar #3369) coated with 0.2 μg/well JRFL in 0.1M sodium bicarbonate and blocked with assay diluent (PBS containing 4% (w/v) whey protein/15% Normal Goat Serum/0.5% Tween20/0.05% Sodium Azide). All assay steps were conducted in assay diluent (except substrate step) and incubated for 1 hour at room temp (13H11 assay at 37°) followed by washing with PBS/0.1% Tween-20. Sera were diluted 1:50 and incubated in triplicate wells. 50 μl biotinylated target Mab was added at the EC50 (determined by direct binding curve of biotinylated-Mab to JRFL). The extent of biotin-Mab binding was detected with streptavidin-alkaline phosphatase at 1:1000 (Promega V5591) followed by substrate (CBC buffer+2 mM MgCl2+1 mg/ml p-npp [4-Nitrophenyl phosphate di(2-amino-2-ethyl-1,3-propanediol) salt]). Plates were read with a plate reader at 405 nm, 45 minutes. Triplicate wells were background subtracted and averaged. Percent inhibition was calculated as follows: 100−(sera triplicate mean/no inhibition control mean)*100. CD4 binding site blocking assays were conducted as above, except that 100 μl of a saturating concentration soluble CD4 (Progenics Pharm Inc.) was incubated between serum and biotin-Mab incubation steps

### AtheNA Assay

Antibody binding to proteins 160, 120, 66, 55, 41, 31, 24 and 17 was measured on the Luminex platform (Luminex Corporation) using the AtheNA Multilyte HIV-1 Bead Blot kit (Zeus Scientific cat #A71001G) following the kit manufacturer's protocol.

### Cardiolipin and Rheumatoid Factor Assays

Anti-cardiolipin antibody assays were performed as described (2). Assays to measure IgM rheumatoid factor using IgG antigen were standardized using rheumatoid factor controls (kindly provided by Judy Fleming, Clinical Immunology Laboratory, Duke University Medical Center).

### Isotype Binding Antibodies

HIV antigens or purified IgM, IgG, IgA proteins (used as controls) were pre-coated overnight onto the wells of microtiter plates (NUNC), washed with an automated and calibrated plate washer (Bio-Tek). The serum/plasma test samples were diluted and incubated with the antigens bound to the plate. The plates were then washed and the antigen-antibody complexes were incubated with isotype specific anti-human IgG, IgA, IgM conjugated to alkaline phosphatase. Optical density readings are measured using a VersaMax plate reader (Molecular Devices) and an average O.D. reading for each pair of replicates, with the background subtracted, was calculated. For each test sample, duplicate antigen-containing and non-antigen-containing wells of a microtiter plate were scored (i.e., O.D. antigen-O.D. non-antigen). A positive score is defined as ≧0.1 O.D., with background subtracted, and also ≧3 fold over baseline with a 15% CV between replicates. As another level of validation, in the plasma donor samples, the HIV gp41 specific IgM binding antibody test was compared with that of the third generation EIA (Abbott Diagnostics, Abbott Park, Ill., USA) and equal sensitivity to the commercially available kit was found for the first detection of any antibody response.

### Specimen Prep MultiTrap IgG Removal.

For detection of IgA and IgM antibodies, IgG was removed using Protein G columns. Briefly, plasma was centrifuged (10,000×g) for 10 minutes, diluted 2-fold in dilution buffer, and filtered in a 1.2 um filter plate (Pall AcroPrep). The filtered and diluted samples were depleted of IgG using a Protein G HP MultiTrap Plates (GE, Inc.) according to manufacturer's instructions with minor modifications. IgG removal in the specimens was greater than 90% as assayed by HIV specific binding assays.

### Customized Luminex Assay

5×10⁶ carboxylated fluorescent beads (Luminex Corp, Austin, Tex.) were covalently coupled to 25 μg of one of the purified HIV antigens used in ELISA assays and incubated with patient samples at a 1:10 dilution. HIV-specific Ab isotypes were detected with goat anti-human IgA (Jackson Immunoresearch, West Grove, Pa.), mouse-anti human IgG (Southern Biotech, Birmingham, Ala.), or goat-anti human IgM (Southern Biotech, Birmingham, Ala.), each conjugated to phycoerythrin, at 4 μg/ml. Beads were then washed and acquired on a Bio-Plex instrument (Bio-Rad, Hercules, Calif.). Purified IgM, IgG, IgA proteins (Sigma) and a constant HIV+ sera titration were utilized as positive controls in every assay. Background values (beads in the absence of detection Ab) and normal human plasma were utilized as the negative controls. A control for Rheumatoid Factor for IgM detection was internal IgG protein standard.

### HIV-1 Immune Complex Capture Assays

ELISA plates (NUNC) were coated overnight at 4° C. with anti-human IgM or IgG at a concentration of 1 μg/ml diluted in phosphate-buffered saline (PBS). All subsequent steps were performed at room temperature. After incubation and washing, coated plates were blocked for 2 h with PBS supplemented with 5% FBS, 5% milk, 0.05% Tween. After blocking and washing, 90 μl undiluted plasma was added to each well and incubated for 90 min, followed by 4 washes with PBS supplemented with 0.05% Tween. 200 μl AVL lysis buffer with carrier RNA was added and shaken for 15 minutes and viral RNA in the lysis was extract by QIAGEN viral mini kit. HIV-1 RNA from the virion-antibody complexes were measured by gag real-time RT-PCR. The detection of immune complexes by the ELISA capture assay was confirmed using Protein G column absorption (Protein G HP, Pierce, Inc) to deplete IgG-virion immune complexes. IgG absorption was performed according the manufacturer's instructions. 90 μl plasma was added to the Protein G column. After mix and incubation of 10 minutes, the column was centrifuged 1 minute at 5000×g. The presence of immune complexes was calculated by the percentage of viral RNA input divided by viral RNA flow through similar to the method by Baron et al. (16). HIVIG (NIH, DAIDS Reagent) plus HIV-1 NL4-3 pseudotyped virus was the positive control for immune complex capture (81±4%), and normal human serum (Sigma) or RPMI-1640 plus HIV-1 NL4-3 was the negative control. The cutoff of non HIV-1 specific capture (normal human serum plus NL43) was 16.2±0.8%, the background of virus only control was 6.5±4.6%.

### Complement Binding Assays

Virus and diluted plasma samples (1:40) were incubated at 37° C. in the presence of 10% Normal Human serum (Sigma, St Louis, Mo.) as a source of complement or with 10% heat inactivated NHS. MT-2 cells which express high levels of CR2 were then added and the Virus/cell suspensions were incubated for 2 hours. Unbound virions were removed by successive washes. Bound virions were disrupted by treatment with 0.5% triton X and the released P24 was measured by ELISA. To determine % binding, the P24 obtained was divided to the P24 of original virus after correcting for complement non specific binding (hi NHS).

### Neutralizing Antibody Assays

Antibody mediated neutralization in the plasma donor cohort was measured as a function of reductions in luciferase reporter gene expression after a single round of infection in TZM-bl cells as described (37). For assay of plasma for 2F- and 4E10-specific neutralizing antibodies, HIV-2 pseudoviruses expressing HIV-1 2F5 or 4E10 epitopes were used as described previously (24).

### Statistical Analyses and Methods to Classify Simultaneous and Sequential Kinetics

Statistical analyses were conducted using methods including mixed effects models (55, 58, 59), nonparametric regression (25), binomial test, Kaplan-Meier curve and accelerated failure time (AFT) model (13). For all four cohorts, smoothing spline based nonparametric regression (25) was performed to obtain estimates of the viral load and antibody curves. For the plasma donor cohort where the acute burst of viremia was recorded, an accurate time origin (T₀) is defined to align different study panels for the joint analysis. For each patient, the T₀ was estimated as a model parameter in the nonlinear mixed effects model of the upswing viral loads, accounting for censoring at the assay limits of detection (55). Right censoring was used in the survival analysis and is defined as ‘No event occurred during the subject's follow up period (while the event could happen at a later time (‘right’ in the time scale). Two subjects were censored because of their short follow up period (12 days post T₀). For each analyte (e.g. anti IgA/IgG/IgM gp41 antibody response), data recorded prior to T₀ were fit to a linear mixed effects model (58) to determine the background level for that analyte, where the upper 95% prediction limit of a future response (59) was used as a positivity threshold to define the last negative observation and the first positive observation. The statistical method of classifying simultaneous and sequential kinetics verified the results obtained from ELISA calculations based on the positivity criterion.

Kaplan-Meier estimate was used to describe the distribution of the initial elevation timing. A two-sided Binomial test of relative ranking in elevation timing between pairs of analytes was performed with a positive difference in timing as a success and the number of non-zero differences as the number of trials. Adjusted p-values (q-values) were computed to control for the false discovery rate (FDR) of multiple testing (54). To assess association between the viral loads and antibody markers in both the plasma donors and CHAVI 001 cohorts, accelerated failure time (AFT) models (13) were used to correlate the expansion or decay of the viral loads and the time to initial elevation (subject to censoring), and linear mixed effects models (58) were used to correlate the downswing viral loads and antibody response magnitudes over time. Additionally, statistical correlation and linear regression analysis were performed to identify the plausible association between different inhibition assays in the Trinidad and CAPRISA cohorts.

### Modeling

The target-cell limited model used to mathematically model the plasma donor VL data is

$$\frac{d T}{d t} = λ - d T - k VT \\ \frac{d l}{d t} = k VT - δ 1 \\ \frac{d V}{d t} = pI - cV \qquad ( 1 )$$

Cells that are susceptible to HIV infection are termed target cells, T. The model assumes that target cells are produced at a constant rate λ and die at rate d per cell. Upon interaction with HIV these cells become productively infected cells, I, with infection rate constant k. Infected cells die at per cell rate δ and produce viral particles (virions), V, at rate p per infected cell. Virions are assumed to be cleared at a constant rate c per virion.

To incorporate enhanced virion clearance due to opsonization, the equation for V(t) was replaced in the model above with the equation

$$\frac{d V}{d t} = pI - c ( 1 + α lg ( t ) ) V \qquad ( 2 )$$

Here virion clearance is enhanced by a factor (1+αIg(t)), where Ig(t) is either the measured concentration of anti-gp41 IgM, anti-gp41 IgG or the total of both immunoglobulin concentrations in plasma. If α=0 then there is no opsonization effect.

To model the effects of antibody in neutralizing virus, the infectivity constant k in Eq. (1) was reduced by the factor (1+β Ig(t). Here, if β=0, there is no antibody mediated viral neutralization. Lastly, to incorporate the possibility of antibody enhancing the rate of infected cell loss through antibody-dependent cellular cytoxicity or complemented mediated lysis, δ was increased by the factor (1+γ Ig(t)). If γ=0 there is no enhanced death.

At T₀, which was chosen as time t=0, the plasma VL by definition is 100 copies/ml. While some CD4+ depletion could have occurred by T₀ for simplicity the uninfected cell level is assumed to still be 10⁶ cells/ml. The number of infected cells at T₀ was estimated as either 1 or 10 cells/ml based on preliminary fits. This low number of infected cells supports the assumption of little T cell depletion by T₀.

The target-cell limited model as well as the three variants of it that included antibody effects were fit to VL data of each plasma donor using a spline fit to the measured anti-gp41 concentrations for Ig(t). Fitting to the VL data was done using a nonlinear least-squares method where logₑ V from the model was fit to the logₑ of the measured VL. An F-test was used to determine whether the target-cell limited model or one of the three variants fit the data best. For donor 9032 the best-fit target-cell limited model gave an extremely poor fit to the data unless a penalty function was added to the sum of squared residuals for not attaining a maximum at the time the VL was maximum. That is, an additional term is added to the function to be minimized equal to the square of the difference between the time the VL was maximum in the data and in the model.

Development of MN Neutralizing Antibodies in Select Subjects from the Trinidad Cohort—Neutralization Assays: cMAGI Assay.

Assays were peformed as previously described (Doria-Rose et al, J. Med. Primatol. 32:218-228 (2003)) with minor modifications. Briefly, serial dilutions of sera were incubated with virus for 1 hour and then added to duplicate wells of cMAGI cells. Infectivity was assessed after 2 days, cells by measuring β-galactosidase expression in fixed and stained cells. Percent neutralization was calculated by [(Vo−Vn)/Vo]×100, where Vn is virus infectivity in the presence of antibodies and Vo is virus infectivity in the absence of antibody. Virus HIV-1_{MN} was obtained through the AIDS Research and Reference Reagent Program, Division of AIDS, National Institute of Allergy and Infectious Diseases, National Institutes of Health.

### Results

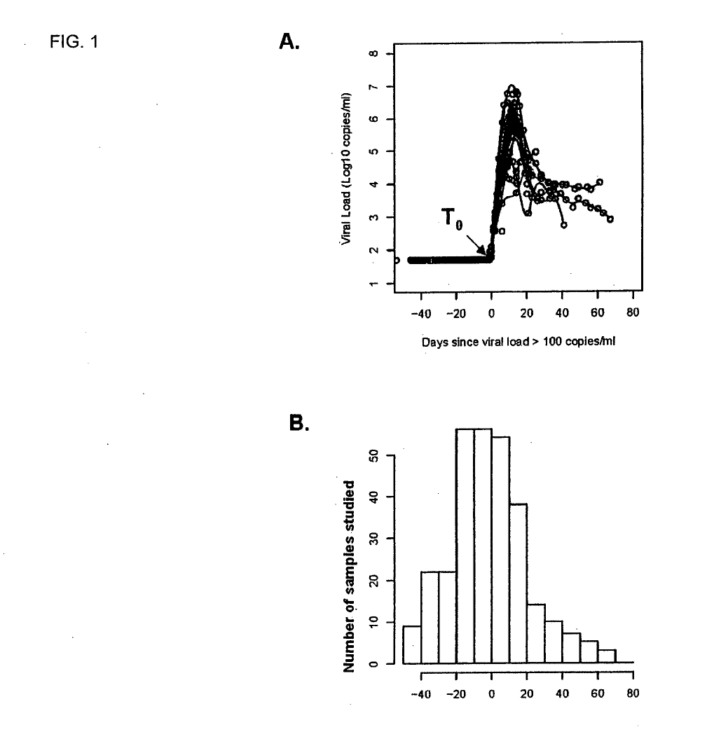

Plasma Donor Cohort. HIV-1 seroconversion plasma donors from the US (Clade B) were studied for the earliest antibody events in HIV-1 infection. These subjects donated plasma every three days and the plasma units were held for weeks until tests for HIV-1, hepatitis B or hepatitis C were completed (19). Once positive for an infectious disease, the plasma sample donations were stopped; therefore, neither cells nor long-term follow-up of these plasma donors were available. Analysis of the US Plasma Donor cohort provided for calculation of the earliest HIV-1 antibody response in relation to a defined point where viral RNA was first detected at transmission. A time zero (T₀) that represented the initial time at which the VL trajectory crossed the assay lower limit of detection (100 HIV-1 copies/ml) was established to align each donor panel to a single reference time (FIG. 1A). The start of detectable plasma viremia, T₀, is approximately 10 days (range 7-21 days) (7, 11, 14, 21, 40, 52) after virus transmission and represents the end of the eclipse phase of HIV-infection. The plasma samples studied from this cohort had the most frequent sampling for testing within the first 20 days before and the first 20 days after the start of detectable viremia (FIG. 1B).

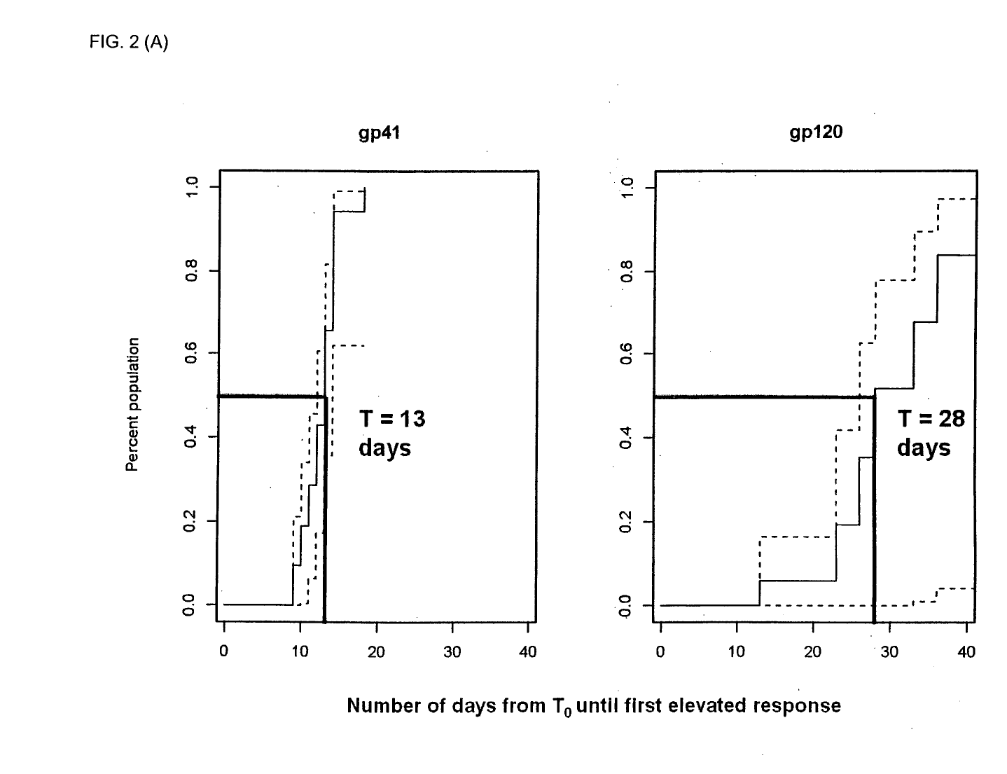

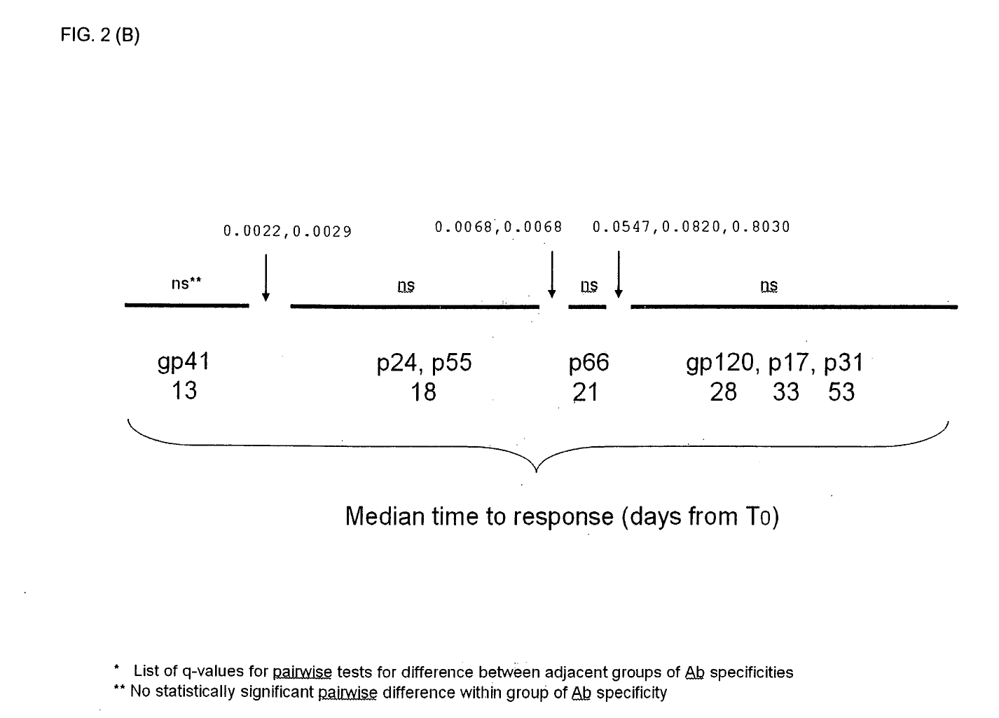

The initial IgG anti-Env responses following transmission. To ensure that the earliest antibodies were detected, both standardized ELISAs and more sensitive Luminex quantitative antibody assays were utilized. The lower level of sensitivity for the ELISAs for measurement of anti-HIV-1 Env IgG was 2.2 ng/ml and the lower level of sensitivity of Luminex assays for measurement of anti-HIV-1 IgG was 0.2 ng/ml. For identification of initial antibody responses, autologous, consensus B and clade B wildtype Envs were tested as antigens. For non-Env antigens, all antigens were wildtype clade B. To validate that consensus B Env antigens detected the earliest antibodies, and to determine if earlier antibody responses could be detected using autologous Env antigens, gp140, and Env gp120 V3 peptides from 4 plasma donor subjects were produced and compared with consensus B Envs or V3 peptides for ability to detect plasma Env antibodies. First, using consensus or wildtype clade B Envs, the earliest detectable anti-Env IgG plasma antibody responses following HIV-1 transmission were found to be to envelope gp41 and occurred at a median of 13.0 days after T₀. FIG. 2A illustrates the earlier timing of the anti-gp41 antibody responses compared to the later and more variable timing of the antibody responses against gp120 (p<0.01). Antibodies to gp41 developed in 90% of subjects by 18 days (KM estimate is 100%, 2 subjects were censored because they were lost to follow up at 12 days post T₀) in contrast to gp120 antibody responses, which came up in 33% of subjects during the followup period of between 12 and 67 days post T₀ studied here (Table 1). There was no significant difference in the timing of the anti-gp120 antibody responses when two additional wildtype clade B gp120 envelope proteins: JRFL and 89.6 gp120 Envs were examined (not shown). FIG. 2B shows the median time of appearance of gp41 and gp120 antibodies compared to time of appearance of antibodies to HIV-1 p24, p55, p66, p17 and p31 HIV-1 proteins. Pair-wise comparison of the timing of each specificity of antibody demonstrated that HIV-1 structural component antibody timing (anti-Gag) was significantly later from that of HIV-1 gp41 antibodies.

A summary of the timing of various anti-Env antibody responses detected in US plasma donors is shown in Table 1. For the first antibody elicited against gp41, 13/19 (68%) of gp41 responses included the immunodominant region of gp41, and 7/7 of the plasma donor initial gp120 included responses that could be mapped to the V3 loop (i.e. 100% of the plasma donors that had gp120 antibodies within the first 40 days of transmission also had V3 antibodies). Antibodies that did not appear at all in US plasma donor subjects (within 40 days post T₀) were anti-MPER (neutralizing and non-neutralizing) antibodies, CD4 binding site antibodies (CD4bs) and CD4-induced antibodies (CD4i). Neutralizing antibodies to the easily neutralized Tier 1 (37) HIV-1 Env pseudoviruses such as B.SF162 and antibody dependent cell-mediated virus inhibition (ADCVI) activity (29) also did not appear within the first 40 days after T₀ as well (data not shown). ADCVI has previously been reported to be present during the later stages of acute infection, so the time points examined here during acute viremia in the plasma donor subjects are likely just before the development of ADCVI (20). As expected, broadly neutralizing antibodies with specificities similar to the broadly neutralizing antibodies 2F5, 4E10, 1b12 and 2G12, as measured by a competitive ELISA with biotinylated monoclonal antibodies, also did not appear during the first 40 days after T₀ (not shown).

Analysis of isotype-specific gp41 antibody responses. IgG antibodies are produced after immunoglobulin (Ig) class switching and are classically produced after IgM antibodies. HIV Env specific IgM was assayed for using Luminex assays with recombinant gp41, gp120 and consensus B and group M consensus gp140 protein antigens. As with IgG responses, the first IgM antibody against Env also only targeted gp41. The median time of rise in HIV-1-specific IgM antibodies was 13 days post T₀ (range 5-18 days). The custom anti-IgM ELISA utilized in this study was equally sensitive to the 3rd generation commercial ELISA (Abbott Anti-HIV ½ EIA, Abbott Park, Ill.) for detection of the first free HIV specific antibody. The difference in timing of first antibody detection between the two assays was not significant, (median difference in days to detection=0, p value=0.66, Wilcoxon signed rank test).

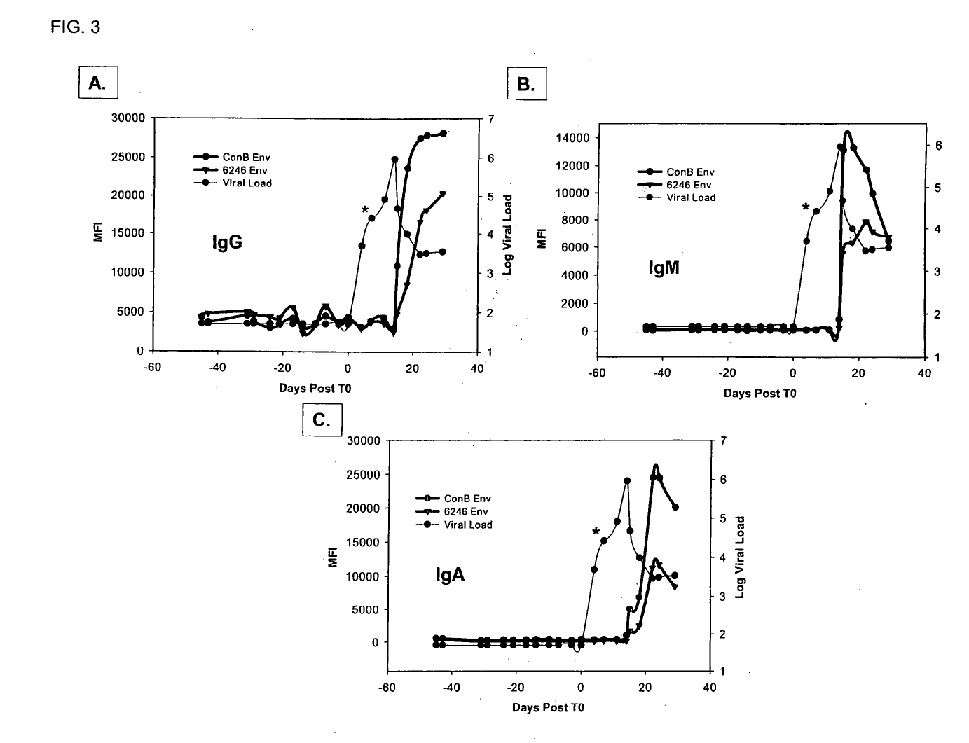

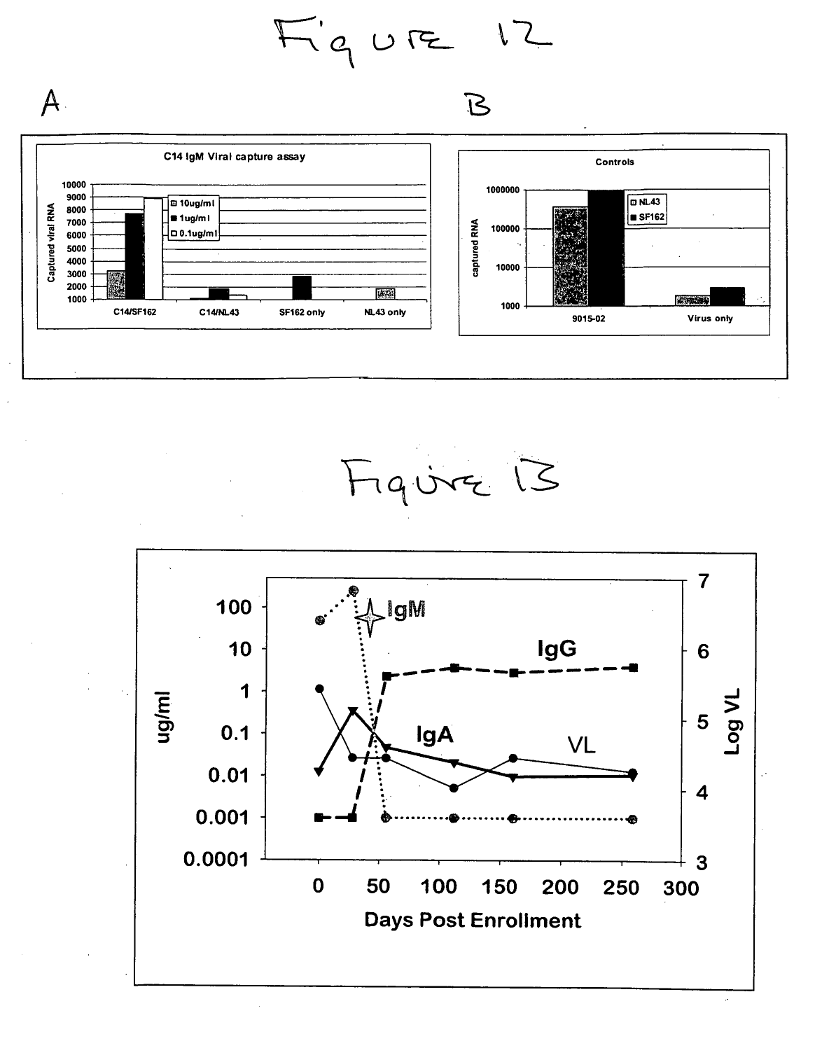

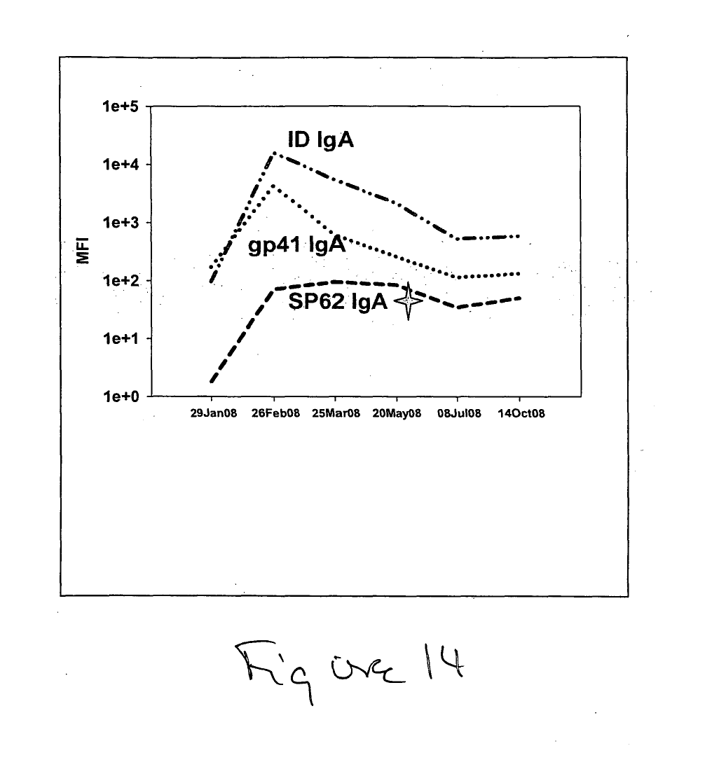

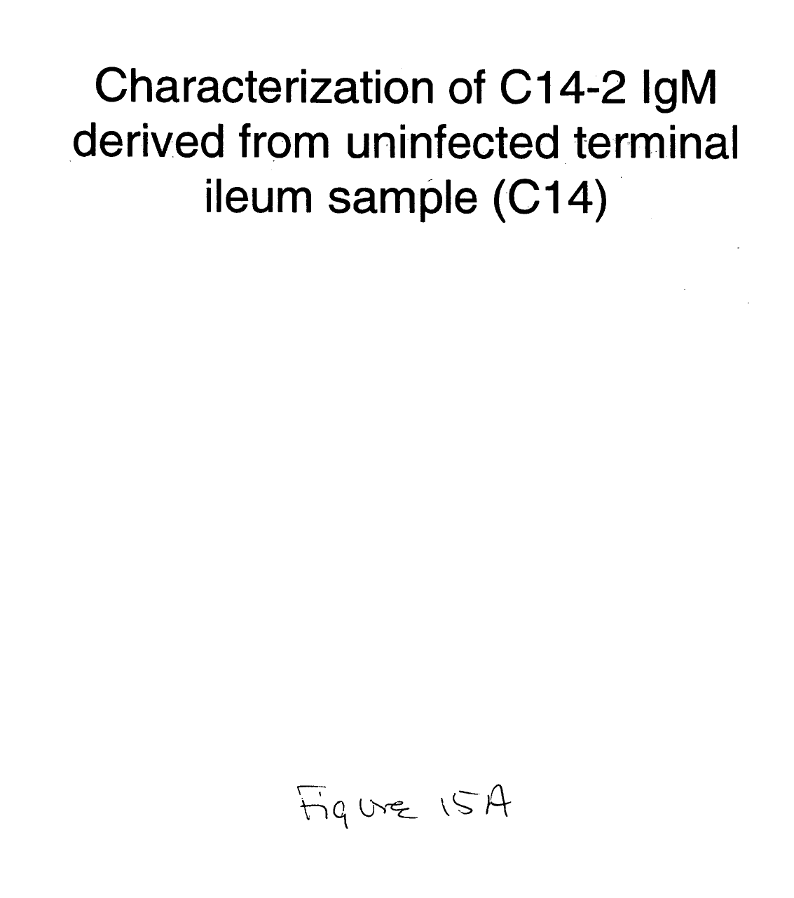

Four autologous gp140 envelopes were expressed, from 4 different plasma donor subjects (Pts. 6246, 6240, 9021, 63521) representing the transmitted or founder virus (35) as gp140C protein oligomers and 4 subjects were studied with autologous Env V3 loop peptides as targets for plasma antibody binding assays. Three of the gp140 Env were chosen from subjects in whom an antibody response was detected with consensus Env, while gp140 was expressed from one subject who did not have a detectable anti-gp41 response. A representative example from a single donor against the autologous and consensus clade B Env (ConB) for IgM, IgG and IgA is shown (FIGS. 3A-3C, and FIG. 10). Using both autologous Envs and autologous V3 peptides (not shown) it was not possible to detect any earlier IgM, IgG and IgA antibody responses compared to those detected using group M consensus Envs or consensus B V3 peptides (FIGS. 3A-3C). Interestingly, the gp41 antibody responses were greater in magnitude when tested with consensus Envs vs. autologous envelopes.

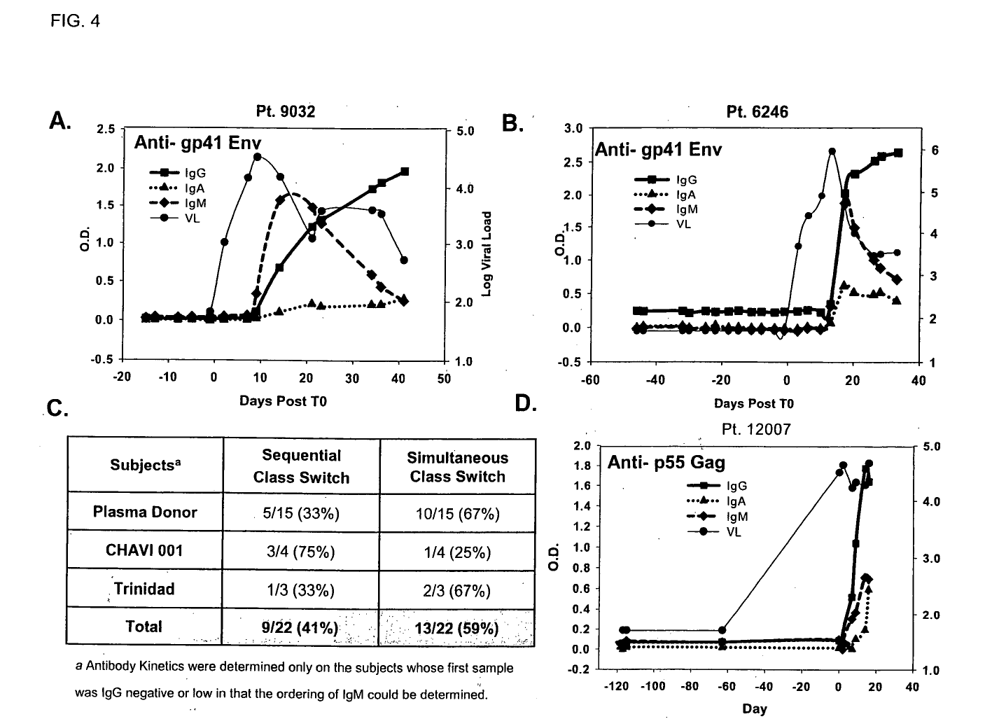

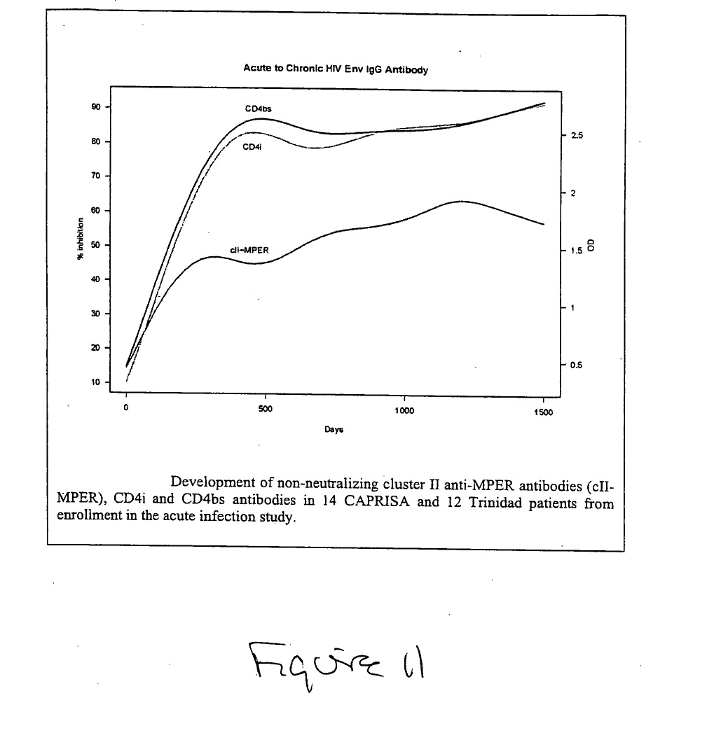

Ig class switching patterns were examined in plasma donors (15 subjects), four clade B CHAVI 001 subjects, and in 3 clade B AHI subjects from the Trinidad/Tobago cohort where the plasma sampling was sufficiently early to determine the first appearances of anti-gp41 IgM and IgG (see Experimental Details; FIG. 9). FIG. 4 shows representative subjects with either sequential class-switch kinetics (FIG. 4A) or simultaneous class-switch kinetics (FIG. 4B) in the plasma donor cohort. In both subjects, IgM responses were transient and decayed over a period of 20-40 days, while IgG responses rose over the same period. Anti-gp41 IgM responses appeared earlier than IgG responses in 9/22 (41%) of subjects; however, in 13/22 (59%) of subjects, IgM anti-gp41 was detected at the same time of IgG and IgA anti-gp41 antibodies (FIG. 4C). Anti-IgM, IgG and IgA responses to an gp41 immunodominant peptide were also tested in subject 6246 with simultaneous appearance of anti-gp41 IgM, IgG and IgA to determine if the simultaneous appearance of the three isotypes could potentially be due to responses to different gp41 epitopes. Anti-immunodominant IgM, IgG and IgA were simultaneously detected at 10 days post T₀ demonstrating in this subject that the simultaneous appearance of antibodies could not solely be attributable to the development of antibody responses to multiple gp41 epitopes (not shown). To determine if simultaneous IgM, IgA and IgG antibody responses were unique to envelope or rather occurred in the response to other HIV-1 proteins, the pattern of isotype antibody responses to p55 Gag was also determined. IgM, IgG and IgA antibodies to p55 Gag were also detected simultaneously in subjects that had simultaneous appearance of IgM, IgG and IgA antibodies to gp41 Env (FIG. 4D).

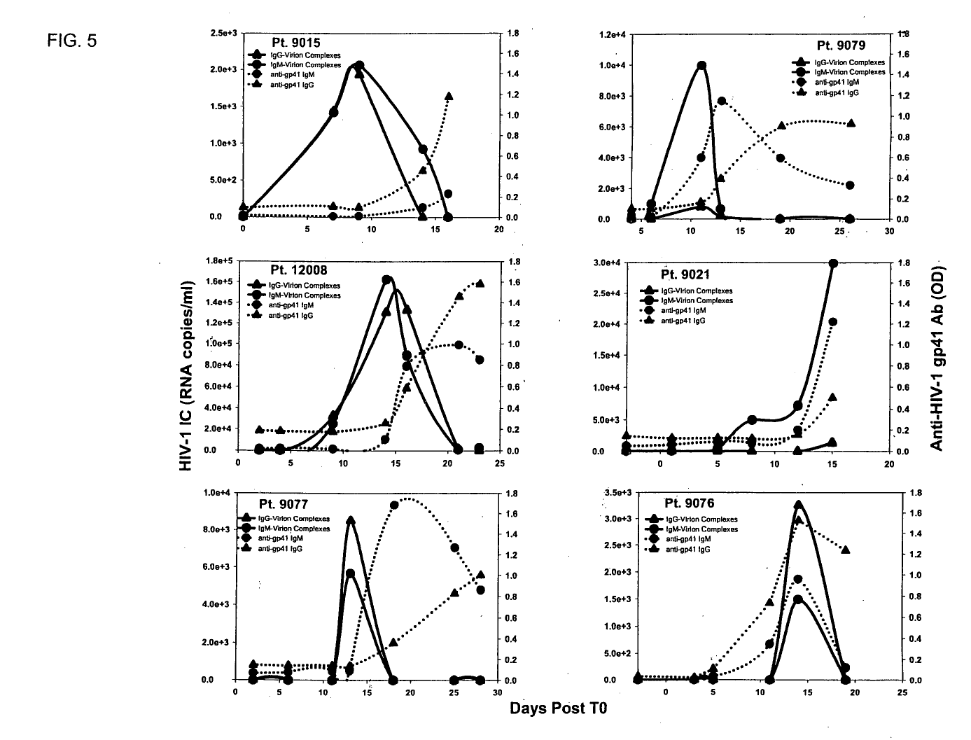

Detection of immune complexes in AHI. US plasma donor panels were assayed for earlier antibody responses in the form of antibody bound to virions to determine if earlier antibody was being made but was not in plasma but rather was only present in the form of virion-IgM or IgG antibody complexes. In 6/6 of subjects tested with sufficiently high plasma viral RNA levels, immune complexes containing either IgG or IgM antibodies bound to virions were detected (FIG. 5). Immune complexes were detected at a median time of 8 days (range: 5-14 days) post T₀. The detection of immune complexes preceded the detection of free antibodies by both commercial ELISA and custom ELISA by a median of 5 days (range 5-7 days) (Table 2). Virion-antibody complexes were detected in subjects with either simultaneous or sequential Ig isotype kinetics, and this suggests that the presence of early immune complexes likely does not explain the simultaneous detection of IgM, IgG and IgA anti-gp4l isotypes.

The detection of IgG immune complexes was confirmed with a second assay of detection (not shown) using a Protein G column to capture antibodies bound to virions followed by lysis of virions to measure viral RNA. Identical kinetics of the appearance and decline of immune complexes were observed using both methods for measurement of IgG immune complexes (not shown). Taken together, these data suggest earlier production of anti-virion IgM and IgG on day 8 after T₀ and before the appearance of free plasma anti-HIV IgM and IgG. The simultaneous appearance of both IgM and IgG virion immune complexes either suggests simultaneous induction of anti-virion IgM and IgG in these subjects or indicates yet earlier induction of IgM and IgG antibodies to HIV virion components with specificities that are not detectable with current assays. The decline in detection of immune complexes may be due to clearance by the reticuloendothelial cell system. It is of interest that the detection of these antibody-virion complexes declines while virus (antigen) and antibody are still present. Further study of the specificities of the antibodies bound in immune complexes and whether they are able to alter infectivity by enabling binding to antigen presenting cells is under investigation.

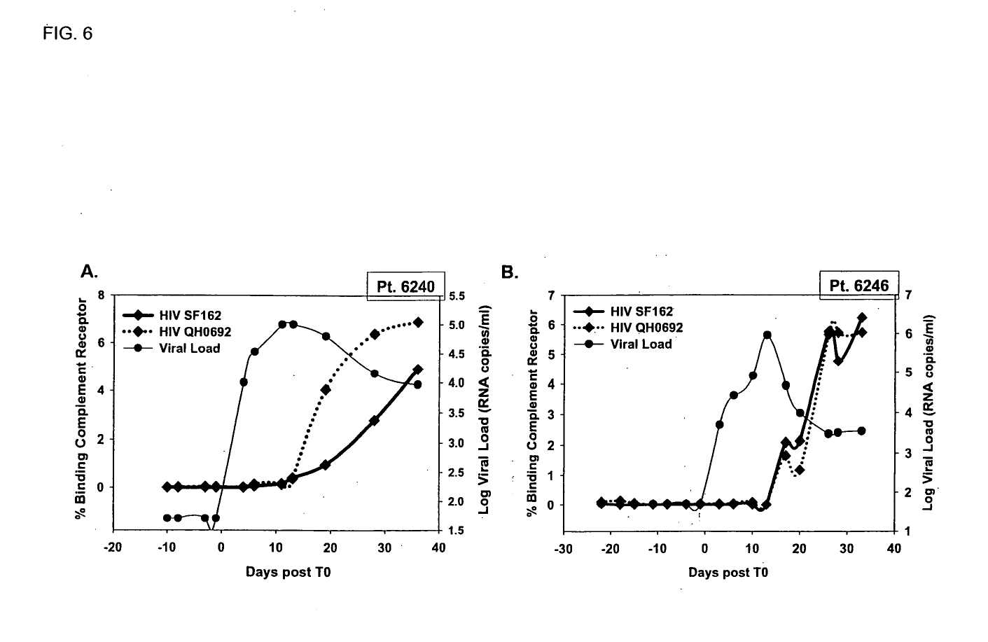

AHI anti-gp41 Env antibodies activate complement. A potentially important functional component of antibodies in AHI is their ability to fix complement. A determination was made as to whether early anti-gp41 antibodies were capable of binding serum complement. Plasmas from 6 US plasma donors were examined for complement activation/binding to CR2 using hPBMC co-cultured with HIV-1 virions. Complement-activating binding antibodies were present in all panels at every time point that plasma antibodies were detected as shown in FIG. 3. Moreover, the kinetics of appearance of complement-activating antibodies followed the same kinetics as gp41 binding antibodies. Both laboratory-adapted HIV-1 strain (B.SF162) and an early transmitted virus strain (B.QH0692) were examined as targets of antibody and complement with similar results obtained with each virus (FIG. 6).

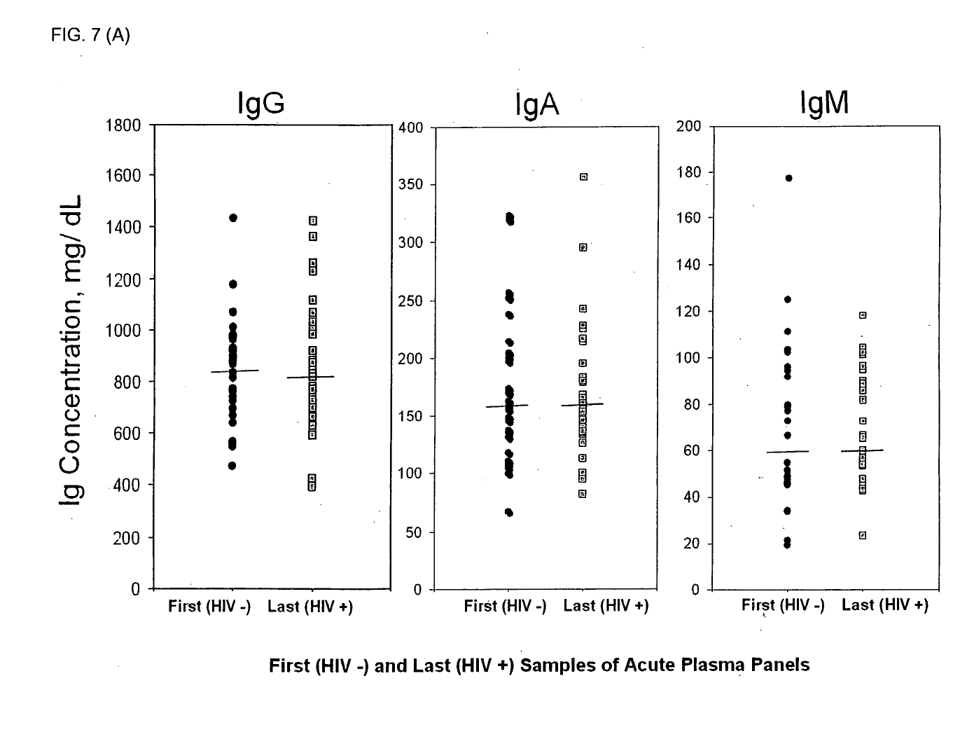

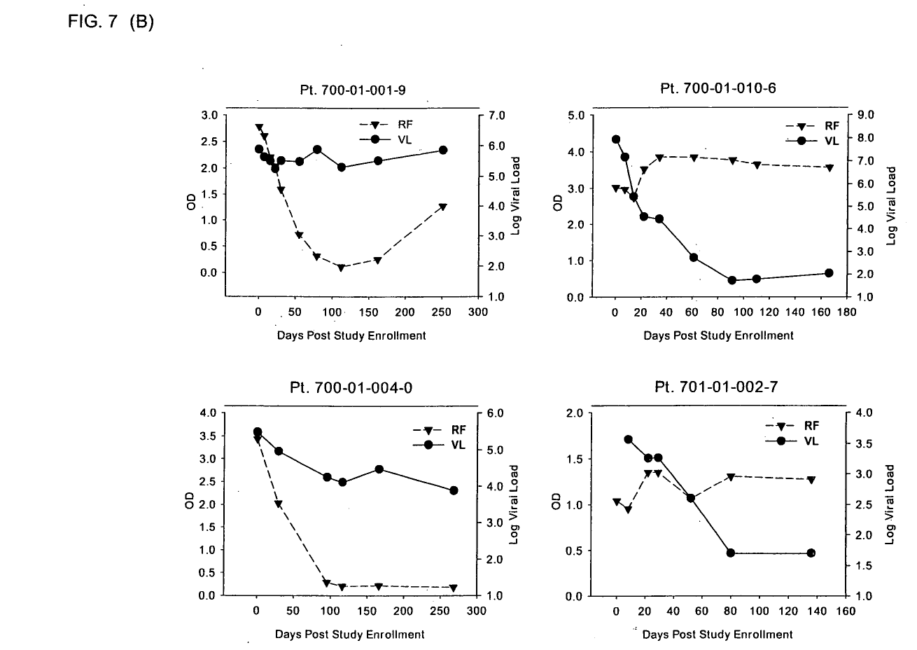

Polyclonal B cell activation following HIV-1 transmission in US plasma donors. HIV-1 Env gp120 has been suggested to be a polyclonal B cell activator (3), to bind to Ig VH3 as a superantigen (23), and to induce polyclonal Ig class switching (28). Patients with chronic HIV-1 infection have polyclonal hypergammaglobulinemia (36), and a number of studies have reported hypergammaglobulinemia in early HIV-1 infection (47, 56). To examine whether polyclonal B cell activation occurs during the initial antibody response to HIV-1 transmitted/founder Env, quantitative IgM, IgG and IgA levels were measured on the initial and last plasma samples in US plasma donors. There was no significant elevation of IgM, IgG or IgA during AHI in plasma donor panels (FIG. 7A). Similarly, the last plasma sample in each donor panel (range of 25-41 days after T₀) was analyzed for the following autoantibodies: cardiolipin, SSA/Ro, SSB/La, Sm, RNP, Scl-70, Jo-1, double stranded DNA, centromere B, and histones and all were negative for all these specificities (not shown). However, in a screen for rheumatoid factor autoantibodies (IgM antibodies that react with IgG) of 19 plasma donor samples and 10 CHAVI 001 acute infection cohort subjects studied, 8/29 (28%) tested positive for rheumatoid factor following HIV-1 transmission (FIG. 7B) with approximately half of those showing a decline in the level of RF antibody after initial detection during acute viremia. Thus, in some subjects, B cell activation during acute HIV-1 infection can result in production of the autoantibody, rheumatoid factor (26).

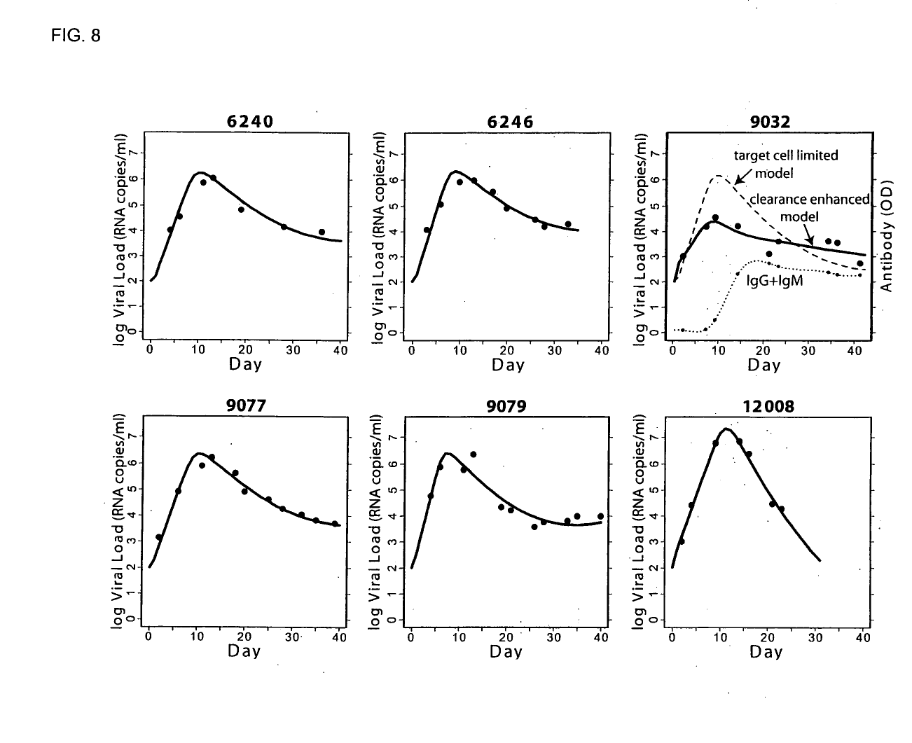

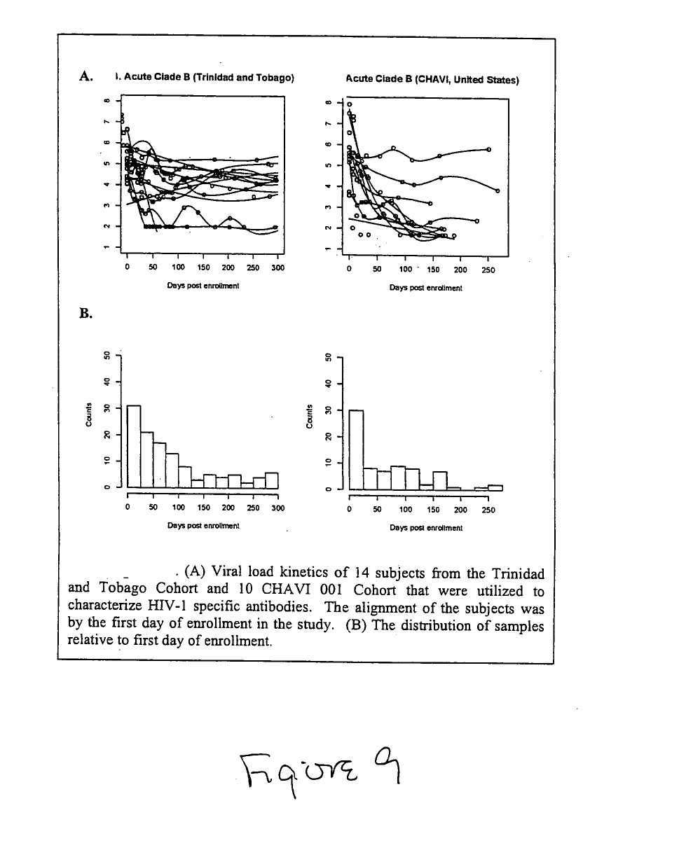

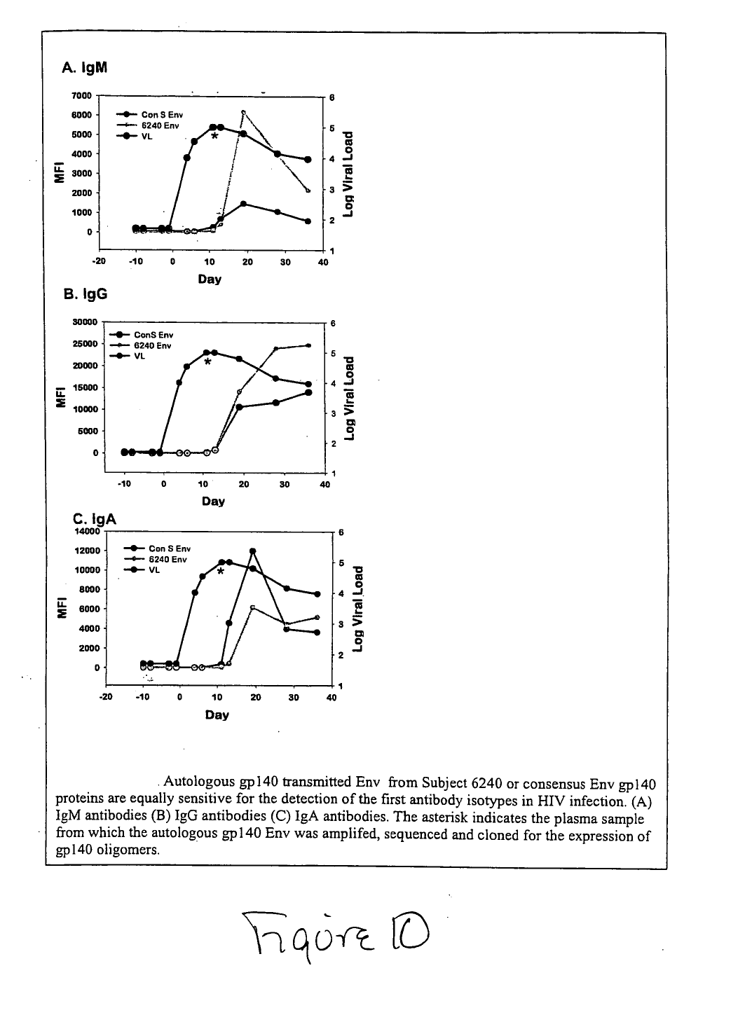

Modeling of the initial gp41 antibody response with acute viral load kinetics and assessment of antibody pressure on viral sequence evolution. To determine the effects of initial anti-gp41 antibody responses on control of HIV-1 viral load (VL), mathematical modeling of the early viral dynamics was used. The target cell-limited model (53) was first used, which does not include any effect of antibody, to analyze the plasma donor VL data obtained over the first 40 days after T₀ for the six donors (6240, 6246, 9032, 9077, 9079 and 12008) for which both complete VL and antibody data were available over this time period. For all donors except 9032, the target cell limited model gave good agreement with the experimentally determined VL data (FIG. 8). Then fit to the same data were three variants of this model that included enhanced virion clearance due to antibody opsonization, antibody-mediated viral neutralization, or antibody-dependent loss of HIV-1 infected cells. In these models (see Experimental Details), the measured levels of anti-gp41 IgM, IgG or the sum of IgM and IgG in mediating these effects were included. Anti-gp120 directed antibodies were not fit in the model since they did not appear in all subjects during the time period examined and when they did, they appeared at a median time of 15 days later than the anti-gp41 antibodies. In all cases, except for donor 9032, including antibody-mediated effects did not improve the model fit (Table 3). Thus, it appears that for the majority of patients the basic target cell-limited model is the most compatible with the data, i.e., including the humoral immune response involving either anti-gp41 IgG, IgM or both does not improve the model fit to the viral kinetics observed for the first 40 days after T₀. This suggests that early in AHI either target-cell limitation or cell-mediated responses play the predominant role in controlling viral load. In support of this notion, no statistically significant association was identified between the magnitude of anti-gp41 IgG and the viral load decay rate. There was also no statistically significant association identified between the viral load decay rate and the time to the first elevation of antibody.

Ontogeny of CD4 inducible (CD4i) antibodies, CD4 binding site antibodies, and non-neutralizing cluster II (MPER) gp4l antibodies in plasma donors and in three additional AHI cohorts followed 6-12 months after transmission. For analysis of antibody responses during the down-slope of VL after transmission, 12 clade B subjects from Trinidad/Tobago with heterosexual transmission, 14 acute clade C subjects from South Africa (the CAPRISA cohort) (24), and 10 clade B US AHI subjects (3 untreated, 7 on anti-retroviral treatment, CHAVI 001 cohort) were studied. The acute phase viral kinetics and distribution of samples used in this study from a subset of subjects in the Trinidad and Tobago and the CHAVI 001 cohort are shown in FIG. 9. Subsequent to the initial B cell response to HIV-1 infection that produces anti-gp41 antibodies, the anti-HIV-1 Env B cell response eventually broadened to include other Env specificities. Antibodies that bind to the MPER gp41 (cluster II antibodies) (63) can either be neutralizing (e.g. Mabs 2F5, Z13, 4E10) or non-neutralizing (e.g. Mabs 267D, 126-6, 13H11) (reviewed in (2)). Whereas non-neutralizing anti-gp41 MPER antibodies are commonly made in ˜80% of infected subjects (2), broadly neutralizing MPER antibodies are rarely made (27). CD4i antibodies bind at or near the co-receptor binding site and potently neutralize HIV-1 generally only after sCD4 is added to the in vitro neutralizing assay (15), due to inability of a bivalent antibody to fit into the coreceptor binding site. Broadly neutralizing CD4BS antibodies are also rarely made (38). Previously described assays for these three anti-Env specificities were used to probe plasma donor panels (followed up to 40 days after T₀) as well as to probe serial plasma samples from selected subjects in the clade C CAPRISA (24) and clade B Trinidad/Tobago (8) acute HIV-1 infection cohorts (both followed 6-12 months after transmission). As mentioned, CD4i, CD4BS, and non-neutralizing cluster II gp41 antibodies were not made during the first 40 days after T₀ (Table 1). In the CAPRISA and Trinidad/Tobago AHI cohorts, CD4i antibodies, CD4 binding site antibodies and non-neutralizing cluster II MPER gp41 antibodies arose at approximately the same time, from 5 to 10 weeks post-enrollment into the acute infection study (FIG. 11) (24).

Evaluation of anti-HIV-1 heterologous Tier 1 and autologous neutralizing antibody responses in plasma donor, CAPRISA and Trinidad AHI cohorts. As previously mentioned, using the highly neutralization-sensitive tier 1 clade B virus, SF162.LS, no neutralizing antibody responses were detected in plasma donors for up to 40 days after T₀. Heterologous tier 1 neutralizing antibodies against HIV-1 MN, were present in the Trinidad/Tobago cohort as early as 8 weeks after infection (Table 4) and were likely primarily V3-directed since autologous V3 peptides competed for heterologous HIV-1MN neutralization (Greenberg, M. L., unpublished). Autologous neutralizing antibodies arose after a median of 32 weeks from the time of transmission in the Trinidad/Tobago clade B cohort (Tomaras, G. D., Greenberg, M. L., unpublished) and at a mean of 19 weeks following transmission in the CAPRISA clade C cohort (24).

In summary, a window of opportunity for immune responses to extinguish HIV-1 exists from the moment of transmission through establishment of the latent pool of HIV-1-infected cells. A critical time to study the initial immune responses to the transmitted/founder virus is the eclipse phase of HIV-1 infection (time from transmission to the first appearance of plasma virus) but, to date, this period has been logistically difficult to analyze. To probe B cell responses immediately following HIV-1 transmission, envelope-specific antibody responses to autologous and consensus Envs have been determined in US plasma donors for whom frequent plasma samples were available at time points immediately before, during, and after HIV-1 plasma viral load (VL) ramp-up in acute infection, and antibody effect modeled on the kinetics of plasma viremia. The first detectable B cell response was in the form of immune complexes 8 days after plasma virus detection, whereas the first free plasma anti-HIV-1 antibody was to gp41 and appeared 13 days after appearance of plasma virus. In contrast, envelope gp120-specific antibodies were delayed an additional 14 days. Mathematical modeling of the earliest viral dynamics was performed to determine the impact of antibody on HIV replication in vivo as assessed by plasma VL. Including the initial anti-gp41 IgG, IgM or both responses in the model did not significantly impact the early dynamics of plasma VL. These results demonstrate that the first IgM and IgG antibodies induced by transmitted HIV-1 are capable of binding virions but have little impact on acute phase viremia at the timing and magnitude that they occur in natural infection.

### EXAMPLE 2

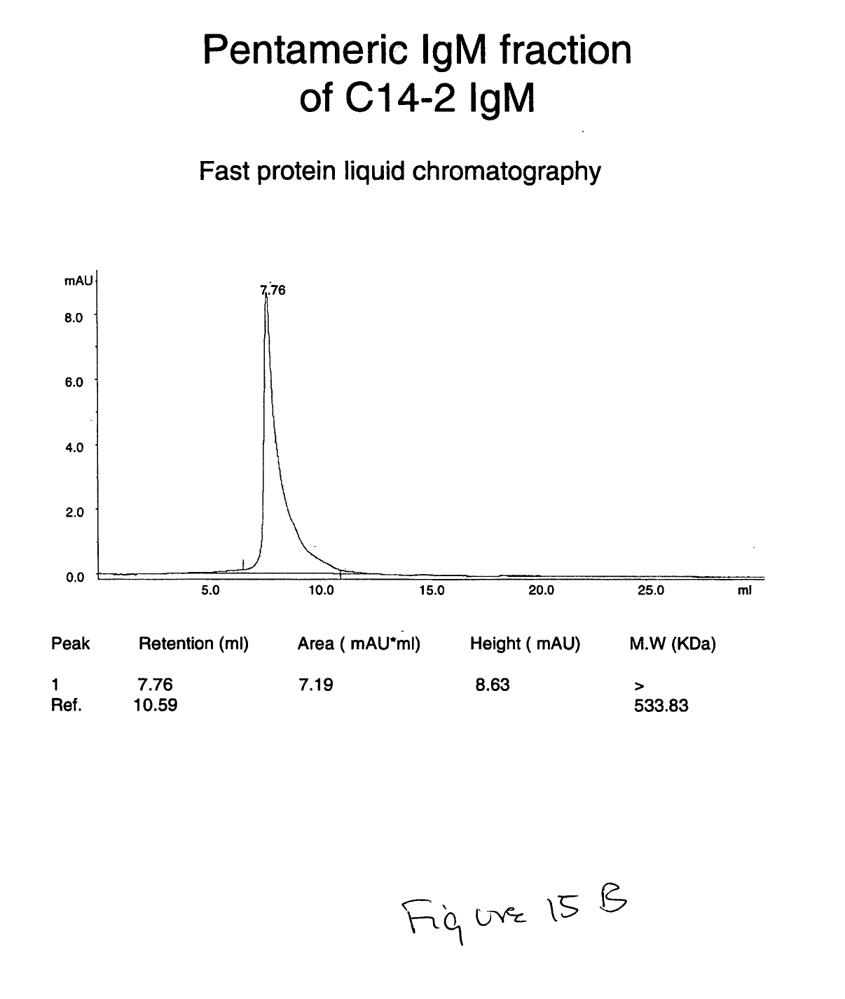

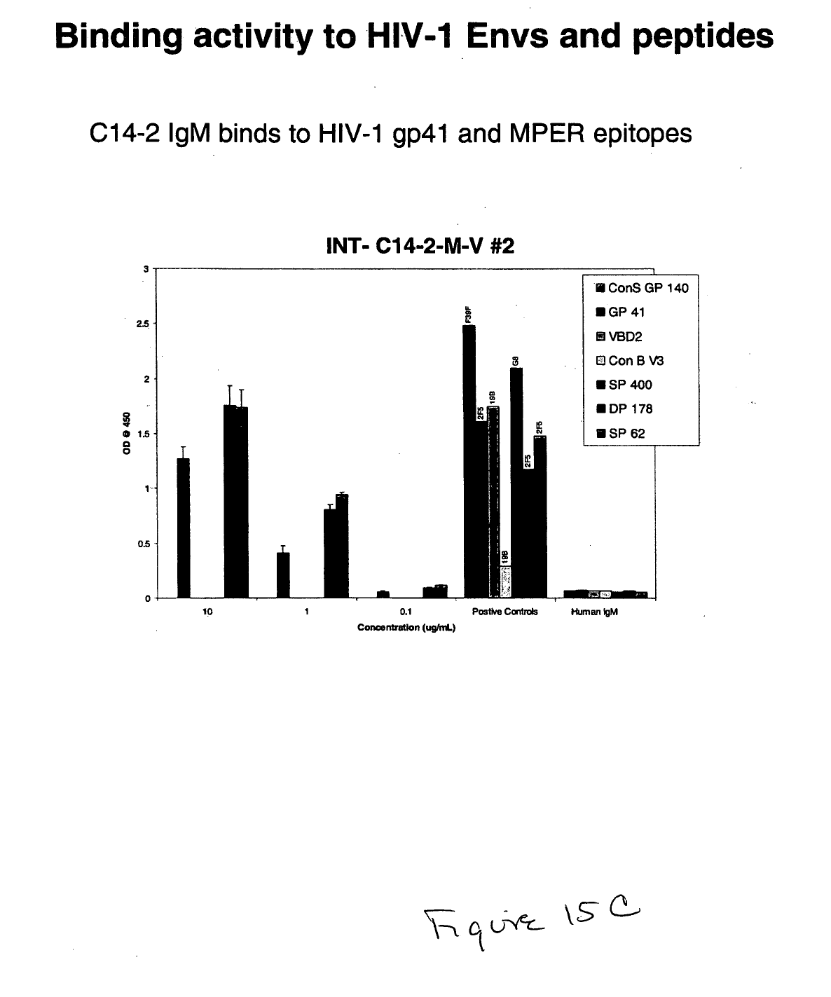

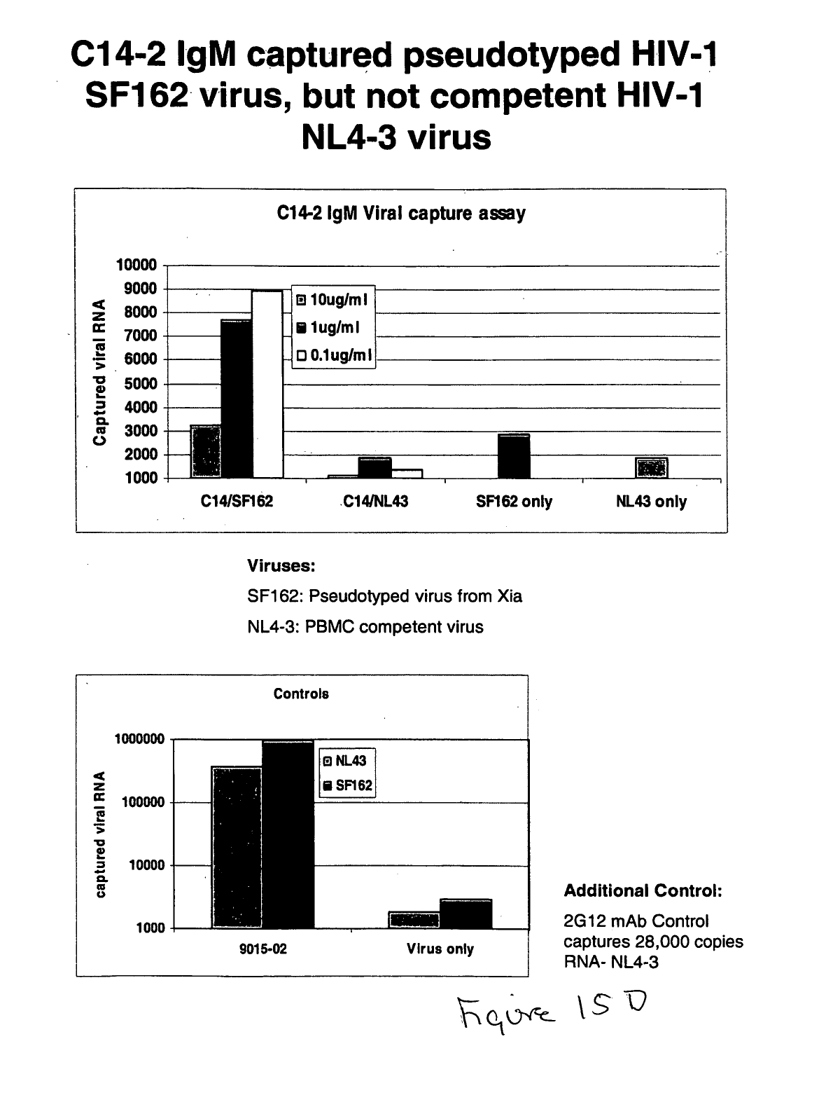

Anti-IgM was coated on to 96 well microtiter plates. Antibody and virus were preincubated together and then applied to the plate. After washing unbound antibody and virus, the amount of antibody-virus complexes captured was determined by quantified viral RNA by RT-PCR. Pseudotyped HIV-1 virus (SF162) and replication competent HIV-1 (NL4-3) were used in this assay. Positive controls for virion capture were the monoclonal antibody 2G12 (copied 28,000 RNA copies of NL4-3) and human plasma positive for virus complexes (9015-02). Negative controls were virus only (SF162 or NL4-3 only) and negative human plasma. As shown in FIG. 12, C14 mAb captured HIV-1 SF162 at an optimal concentration of 0.1 μg/ml of antibody.

### EXAMPLE 3

Seminal plasma from acute HIV-1 subjects were examined for binding to HIV-1 antigens using a custom HIV-1 luminex panel. IgA and IgM are measured from diluted seminal plasma depleted of IgG by high throughput Protein G purification. Binding to antigen is measured in terms of mean fluorescent intensity (MFI) and converted to a concentration measurement (μg/ml) based on a positive control antibody titration with know concentrations.

Envelope HIV-1 specific antibodies are detected in seminal plasma at early times in acute infection. In contrast to HIV-1 specific IgG, HIV-1 Env specific IgA declines during the acute phase of infection. In some individuals, although rare, HIV-1 specific IgM is detected. This subject has unusually high levels (100 μg/ml) of HIV-1 specific IgM antibodies.

### EXAMPLE 4

Seminal plasma from acute HIV-1 subjects were examined for binding to HIV-1 antigens using a custom HIV-1 luminex panel. IgA was measured from diluted seminal plasma depleted of IgG by high throughput Protein G purification. Binding to antigen is measured in terms of mean fluorescent intensity (MFI) and converted to a concentration measurement (μg/ml) based on a positive control antibody titration with know concentrations.

Gp41 Env HIV-1 specific antibodies are detected in seminal plasma at early times in acute infection. In some individuals, IgA antibodies are specific for the 2F5 epitope of the HIV-1 Env MPER, in the absence of detectable IgG antibodies for the same epitope.

### REFERENCES

1. Aasa-Chapman, M. M., A. Hayman, P. Newton, D. Cornforth, I. Williams, P. Borrow, P. Balfe, and A. McKnight. 2004. Development of the antibody response in acute HIV-1 infection. Aids 18:371-81.
2. Alam, S. M., R. M. Scearce, R. J. Parks, K. Plonk, S. G. Plonk, L. L. Sutherland, M. K. Gorny, S. Zolla-Pazner, S. Vanleeuwen, M. A. Moody, S. M. Xia, D. C. Montefiori, G. D. Tomaras, K. J. Weinhold, S. A. Karim, C. B. Hicks, H. X. Liao, J. Robinson, G. M. Shaw, and B. F. Haynes. 2008. Human immunodeficiency virus type 1 gp41 antibodies that mask membrane proximal region epitopes: antibody binding kinetics, induction, and potential for regulation in acute infection. J Virol 82:115-25.
3. Berberian, L., L. Goodglick, T. J. Kipps, and J. Braun. 1993. Immunoglobulin VH3 gene products: natural ligands for HIV gp120. Science 261:1588-91.
4. Brenchley, J. M., T. W. Schacker, L. E. Ruff, D. A. Price, J. H. Taylor, G. J. Beilman, P. L. Nguyen, A. Khoruts, M. Larson, A. T. Haase, and D. C. Douek. 2004. CD4+ T cell depletion during all stages of HIV disease occurs predominantly in the gastrointestinal tract. J Exp Med 200:749-59.
5. Carne, C. A., R. S. Tedder, A. Smith, S. Sutherland, S. G. Elkington, H. M. Daly, F. E. Preston, and J. Craske. 1985. Acute encephalopathy coincident with seroconversion for anti-HTLV-III. Lancet 2:1206-8.
6. Chun, T. W., D. Engel, M. M. Berrey, T. Shea, L. Corey, and A. S. Fauci. 1998. Early establishment of a pool of latently infected, resting CD4(+) T cells during primary HIV-1 infection. Proc Natl Acad Sci USA 95:8869-73.
7. Clark, S. J., M. S. Saag, W. D. Decker, S. Campbell-Hill, J. L. Roberson, P. J. Veldkamp, J. C. Kappes, B. H. Hahn, and G. M. Shaw. 1991. High titers of cytopathic virus in plasma of patients with symptomatic primary HIV-1 infection. N Engl J Med 324:954-60.
8. Cleghorn, F. R., N. Jack, J. K. Carr, J. Edwards, B. Mahabir, A. Sill, C. B. McDanal, S. M. Connolly, D. Goodman, R. Q. Bennetts, T. R. O'Brien, K. J. Weinhold, C. Bartholomew, W. A. Blattner, and M. L. Greenberg. 2000. A distinctive clade B HIV type 1 is heterosexually transmitted in Trinidad and Tobago. Proc Natl Acad Sci USA 97:10532-7.
9. Clutterbuck, E. A., P. Salt, S. Oh, A. Marchant, P. Beverley, and A. J. Pollard. 2006. The kinetics and phenotype of the human B-cell response following immunization with a heptavalent pneumococcal-CRM conjugate vaccine. Immunology 119:328-37.
10. Cooper, D. A., J. Gold, P. Maclean, B. Donovan, R. Finlayson, T. G. Barnes, H. M. Michelmore, P. Brooke, and R. Penny. 1985. Acute AIDS retrovirus infection. Definition of a clinical illness associated with seroconversion. Lancet 1:537-40.
11. Cooper, D. A., A. A. Imrie, and R. Penny. 1987. Antibody response to human immunodeficiency virus after primary infection. J Infect Dis 155:1113-8.
12. Coordinating Committee of the Global HIV/AIDS Vaccine Enterprise. 2005. The Global HIV/AIDS Vaccine Enterprise: scientific strategic plan. PLoS Med 2:e25.
13. Cox, D. R., and D. Oakes. 1984. Analysis of survival data. Chapman and Hall, London; New York.
14. Daar, E. S., T. Moudgil, R. D. Meyer, and D. D. Ho. 1991. Transient high levels of viremia in patients with primary human immunodeficiency virus type 1 infection. N Engl J Med 324:961-4.
15. Decker, J. M., F. Bibollet-Ruche, X. Wei, S. Wang, D. N. Levy, W. Wang, E. Delaporte, M. Peeters, C. A. Derdeyn, S. Allen, E. Hunter, M. S. Saag, J. A. Hoxie, B. H. Hahn, P. D. Kwong, J. E. Robinson, and G. M. Shaw. 2005. Antigenic conservation and immunogenicity of the HIV coreceptor binding site. J Exp Med 201:1407-19.
16. Dianzani, F., G. Antonelli, E. Riva, O. Turriziani, L. Antonelli, S. Tyring, D. A. Carrasco, H. Lee, D. Nguyen, J. Pan, J. Poast, M. Cloyd, and S. Baron. 2002. Is human immunodeficiency virus RNA load composed of neutralized immune complexes? J Infect Dis 185:1051-4.
17. Douek, D. C., P. D. Kwong, and G. J. Nabel. 2006. The rational design of an AIDS vaccine. Cell 124:677-81.
18. Emau, P., Y. Jiang, M. B. Agy, B. Tian, G. Bekele, and C. C. Tsai. 2006. Post-exposure prophylaxis for SIV revisited: animal model for HIV prevention. AIDS Res Ther 3:29.
19. Fiebig, E. W., D. J. Wright, B. D. Rawal, P. E. Garrett, R. T. Schumacher, L. Peddada, C. Heldebrant, R. Smith, A. Conrad, S. H. Kleinman, and M. P. Busch. 2003. Dynamics of HIV viremia and antibody seroconversion in plasma donors: implications for diagnosis and staging of primary HIV infection. Aids 17:1871-9.
20. Forthal, D. N., G. Landucci, and E. S. Daar. 2001. Antibody from patients with acute human immunodeficiency virus (HIV) infection inhibits primary strains of HIV type 1 in the presence of natural-killer effector cells. J Virol 75:6953-61.
21. Gaines, H., M. von Sydow, A. Sonnerborg, J. Albert, J. Czajkowski, P. O. Pehrson, F. Chiodi, L. Moberg, E. M. Fenyo, B. Asjo, and et al. 1987. Antibody response in primary human immunodeficiency virus infection. Lancet 1:1249-53.
22. Gasper-Smith, N., D. M. Crossman, J. F. Whitesides, N. Mensali, J. S. Ottinger, S. G. Plonk, M. A. Moody, G. Ferrari, K. J. Weinhold, S. E. Miller, C. F. Reich, 3rd, L. Qin, S. G. Self, G. M. Shaw, T. N. Denny, L. E. Jones, D. S. Pisetsky, and B. F. Haynes. 2008. Induction of Plasma (TRAIL), TNFR-2, Fas Ligand and Plasma Microparticles After HIV-1 Transmission: Implications for HIV-1 Vaccine Design. J Virol.
23. Goodglick, L., N. Zevit, M. S. Neshat, and J. Braun. 1995. Mapping the Ig superantigen-binding site of HIV-1 gp120. J Immunol 155:5151-9.
24. Gray, E. S., P. L. Moore, I. A. Choge, J. M. Decker, F. Bibollet-Ruche, H. Li, N. Leseka, F. Treurnicht, K. Mlisana, G. M. Shaw, S. S. Karim, C. Williamson, and L. Morris. 2007. Neutralizing antibody responses in acute human immunodeficiency virus type 1 subtype C infection. J Virol 81:6187-96.
25. Green, P. J., and B. W. Silverman. 1994. Nonparametric regression and generalized linear models: a roughness penalty approach, 1st ed. Chapman & Hall, London; New York.
26. Hardy, R. R., K. Hayakawa, M. Shimizu, K. Yamasaki, and T. Kishimoto. 1987. Rheumatoid factor secretion from human Leu-1+ B cells. Science 236:81-3.
27. Haynes, B. F., and D. C. Montefiori. 2006. Aiming to induce broadly reactive neutralizing antibody responses with HIV-1 vaccine candidates. Expert Rev Vaccines 5:347-63.
28. He, B., X. Qiao, P. J. Klasse, A. Chiu, A. Chadburn, D. M. Knowles, J. P. Moore, and A. Cerutti. 2006. HIV-1 envelope triggers polyclonal Ig class switch recombination through a CD40-independent mechanism involving BAFF and C-type lectin receptors. J Immunol 176:3931-41.
29. Hessell, A. J., L. Hangartner, M. Hunter, C. E. Havenith, F. J. Beurskens, J. M. Bakker, C. M. Lanigan, G. Landucci, D. N. Forthal, P. W. Parren, P. A. Marx, and D. R. Burton. 2007. Fc receptor but not complement binding is important in antibody protection against HIV. Nature 449:101-4.
30. Ho, D. D., M. G. Sarngadharan, L. Resnick, F. Dimarzoveronese, T. R. Rota, and M. S. Hirsch. 1985. Primary human T-lymphotropic virus type III infection. Ann Intern Med 103:880-3.
31. Holl, V., M. Peressin, T. Decoville, S. Schmidt, S. Zolla-Pazner, A. M. Aubertin, and C. Moog. 2006. Nonneutralizing antibodies are able to inhibit human immunodeficiency virus type 1 replication in macrophages and immature dendritic cells. J Virol 80:6177-81.
32. Holl, V., M. Peressin, S. Schmidt, T. Decoville, S. Zolla-Pazner, A. M. Aubertin, and C. Moog. 2006. Efficient inhibition of HIV-1 replication in human immature monocyte-derived dendritic cells by purified anti-HIV-1 IgG without induction of maturation. Blood 107:4466-74.
33. Huber, M., M. Fischer, B. Misselwitz, A. Manrique, H. Kuster, B. Niederost, R. Weber, V. von Wyl, H. F. Gunthard, and A. Trkola. 2006. Complement lysis activity in autologous plasma is associated with lower viral loads during the acute phase of HIV-1 infection. PLoS Med 3:e441.
34. Johnston, M. I., and A. S. Fauci. 2007. An HIV vaccine—evolving concepts. N Engl J Med 356:2073-81.
35. Keele, B. F., E. E. Giorgi, J. F. Salazar-Gonzalez, J. M. Decker, K. T. Pham, M. G. Salazar, C. Sun, T. Grayson, S. Wang, H. Li, X. Wei, C. Jiang, J. L. Kirchherr, F. Gao, J. A. Anderson, L. H. Ping, R. Swanstrom, G. D. Tomaras, W. A. Blattner, P. A. Goepfert, J. M. Kilby, M. S. Saag, E. L. Delwart, M. P. Busch, M. S. Cohen, D. C. Montefiori, B. F. Haynes, B. Gaschen, G. S. Athreya, H. Y. Lee, N. Wood, C. Seoighe, A. S. Perelson, T. Bhattacharya, B. T. Korber, B. H. Hahn, and G. M. Shaw. 2008. Identification and characterization of transmitted and early founder virus envelopes in primary HIV-1 infection. Proc Natl Acad Sci USA 105:7552-7.
36. Lane, H. C., H. Masur, L. C. Edgar, G. Whalen, A. H. Rook, and A. S. Fauci. 1983. Abnormalities of B-cell activation and immunoregulation in patients with the acquired immunodeficiency syndrome. N Engl J Med 309:453-8.
37. Li, M., F. Gao, J. R. Mascola, L. Stamatatos, V. R. Polonis, M. Koutsoukos, G. Voss, P. Goepfert, P. Gilbert, K. M. Greene, M. Bilska, D. L. Kothe, J. F. Salazar-Gonzalez, X. Wei, J. M. Decker, B. H. Hahn, and D. C. Montefiori. 2005. Human immunodeficiency virus type 1 env clones from acute and early subtype B infections for standardized assessments of vaccine-elicited neutralizing antibodies. J Virol 79:10108-25.
38. Li, Y., S. A. Migueles, B. Welcher, K. Svehla, A. Phogat, M. K. Louder, X. Wu, G. M. Shaw, M. Connors, R. T. Wyatt, and J. R. Mascola. 2007. Broad HIV-1 neutralization mediated by CD4-binding site antibodies. Nat Med 13:1032-4.
39. Liao, H. X., L. L. Sutherland, S. M. Xia, M. E. Brock, R. M. Scearce, S. Vanleeuwen, S. M. Alam, M. McAdams, E. A. Weaver, Z. Camacho, B. J. Ma, Y. Li, J. M. Decker, G. J. Nabel, D. C. Montefiori, B. H. Hahn, B. T. Korber, F. Gao, and B. F. Haynes. 2006. A group M consensus envelope glycoprotein induces antibodies that neutralize subsets of subtype B and C HIV-1 primary viruses. Virology 353:268-82.
40. Little, S. J., A. R. McLean, C. A. Spina, D. D. Richman, and D. V. Havlir. 1999. Viral dynamics of acute HIV-1 infection. J Exp Med 190:841-50.
41. Mascola, J. R., M. G. Lewis, G. Stiegler, D. Harris, T. C. VanCott, D. Hayes, M. K. Louder, C. R. Brown, C. V. Sapan, S. S. Frankel, Y. Lu, M. L. Robb, H. Katinger, and D. L. Birx. 1999. Protection of Macaques against pathogenic simian/human immunodeficiency virus 89.6PD by passive transfer of neutralizing antibodies. J Virol 73:4009-18.
42. Mascola, J. R., S. W. Snyder, O. S. Weislow, S. M. Belay, R. B. Belshe, D. H. Schwartz, M. L. Clements, R. Dolin, B. S. Graham, G. J. Gorse, M. C. Keefer, M. J. McElrath, M. C. Walker, K. F. Wagner, J. G. McNeil, F. E. McCutchan, and D. S. Burke. 1996. Immunization with envelope subunit vaccine products elicits neutralizing antibodies against laboratory-adapted but not primary isolates of human immunodeficiency virus type 1. The National Institute of Allergy and Infectious Diseases AIDS Vaccine Evaluation Group. J Infect Dis 173:340-8.
43. Moir, S., and A. S. Fauci. 2008. Pathogenic mechanisms of B-lymphocyte dysfunction in HIV disease. J Allergy Clin Immunol.
44. Montero, M., N. E. van Houten, X. Wang, and J. K. Scott. 2008. The membrane-proximal external region of the human immunodeficiency virus type 1 envelope: dominant site of antibody neutralization and target for vaccine design. Microbiol Mol Biol Rev 72:54-84, table of contents.
45. Moore, J. P., Y. Cao, D. D. Ho, and R. A. Koup. 1994. Development of the anti-gp120 antibody response during seroconversion to human immunodeficiency virus type 1. J Virol 68:5142-55.
46. Moore, P. L., E. S. Gray, I. A. Choge, N. Ranchobe, K. Mlisana, S. S. Abdool Karim, C. Williamson, and L. Morris. 2008. The c3-v4 region is a major target of autologous neutralizing antibodies in human immunodeficiency virus type 1 subtype C infection. J Virol 82:1860-9.
47. Morris, L., J. M. Binley, B. A. Clas, S. Bonhoeffer, T. P. Astill, R. Kost, A. Hurley, Y. Cao, M. Markowitz, D. D. Ho, and J. P. Moore. 1998. HIV-1 antigen-specific and -nonspecific B cell responses are sensitive to combination antiretroviral therapy. J Exp Med 188:233-45.
48. Phogat, S., K. Svehla, M. Tang, A. Spadaccini, J. Muller, J. Mascola, I. Berkower, and R. Wyatt. 2008. Analysis of the human immunodeficiency virus type 1 gp41 membrane proximal external region arrayed on hepatitis B surface antigen particles. Virology 373:72-84.
49. Pinter, A. 2007. Roles of HIV-1 Env variable regions in viral neutralization and vaccine development. Curr HIV Res 5:542-53.
50. Richman, D. D., T. Wrin, S. J. Little, and C. J. Petropoulos. 2003. Rapid evolution of the neutralizing antibody response to HIV type 1 infection. Proc Natl Acad Sci USA 100:4144-9.
51. Scanlan, C. N., J. Offer, N. Zitzmann, and R. A. Dwek. 2007. Exploiting the defensive sugars of HIV-1 for drug and vaccine design. Nature 446:1038-45.
52. Schacker, T., A. C. Collier, J. Hughes, T. Shea, and L. Corey. 1996. Clinical and epidemiologic features of primary HIV infection. Ann Intern Med 125:257-64.
53. Stafford, M. A., L. Corey, Y. Cao, E. S. Daar, D. D. Ho, and A. S. Perelson. 2000. Modeling plasma virus concentration during primary HIV infection. J Theor Biol 203:285-301.
54. Storey, J. D. 2002. A direct approach to false discovery rates. Journal of the Royal Statistical Society 64:479-498.
55. Thiebaut, R., and H. Jacqmin-Gadda. 2004. Mixed models for longitudinal left-censored repeated measures. Comput Methods Programs Biomed 74:255-60.
56. Titanji, K., F. Chiodi, R. Bellocco, D. Schepis, L. Osorio, C. Tassandin, G. Tambussi, S. Grutzmeier, L. Lopalco, and A. De Milito. 2005. Primary HIV-1 infection sets the stage for important B lymphocyte dysfunctions. Aids 19:1947-55.
57. Veazey, R. S., P. M. Acierno, K. J. McEvers, S. H. Baumeister, G. J. Foster, M. D. Rett, M. H. Newberg, M. J. Kuroda, K. Williams, E. Y. Kim, S. M. Wolinsky, E. P. Rieber, M. Piatak, Jr., J. D. Lifson, D. C. Montefiori, C. R. Brown, V. M. Hirsch, and J. E. Schmitz. 2008. Increased loss of CCR5+ CD45RA− CD4+ T cells in CD8+ lymphocyte-depleted Simian immunodeficiency virus-infected rhesus monkeys. J Virol 82:5618-30.
58. Verbeke, G., and G. Molenberghs. 2000. Linear mixed models for longitudinal data. Springer, New York.
59. Vidoni, P. 2006. Response prediction in mixed effects models. Journal of Statistical Planning and Inference 136:3948-3966.
60. Wei, X., J. M. Decker, S. Wang, H. Hui, J. C. Kappes, X. Wu, J. F. Salazar-Gonzalez, M. G. Salazar, J. M. Kilby, M. S. Saag, N. L. Komarova, M. A. Nowak, B. H. Hahn, P. D. Kwong, and G. M. Shaw. 2003. Antibody neutralization and escape by HIV-1. Nature 422:307-12.
61. Wong, S. B. J., and R. F. Siliciano. 2007. Biology of Early Infection and Impact on Vaccine Design, p. 17-22. In P. K. Wayne C. Koff, Ian D Gust (ed.), AIDS Vaccine Development: Challenges and Opportunities. Caister Academic Press.
62. Wu, L., and V. N. KewalRamani. 2006. Dendritic-cell interactions with HIV: infection and viral dissemination. Nat Rev Immunol 6:859-68.
63. Xu, J. Y., M. K. Gorny, T. Palker, S. Karwowska, and S. Zolla-Pazner. 1991. Epitope mapping of two immunodominant domains of gp41, the transmembrane protein of human immunodeficiency virus type 1, using ten human monoclonal antibodies. J Virol 65:4832-8.
64. Xu, W., R. Hofmann-Lehmann, H. M. McClure, and R. M. Ruprecht. 2002. Passive immunization with human neutralizing monoclonal antibodies: correlates of protective immunity against HIV. Vaccine 20:1956-60.

All documents and other information sources cited above are hereby incorporated in their entirety by reference.

TABLE 1
Ontogeny: Env Specific IgG in Eclipse Phase Clade B Cohort.

| | | Median | 0.95 | 0.95 | |
|---|---|---|---|---|---|
| Antigen | N^{a} | Time^{b} | LCL | UCL | Range |
| gp41 | 19 | 13 | 12 | 14 | (9-18) |
| gp140 | 19 | 13 | 12 | 15 | (6-17) |
| ID^{c} gp41 peptide | 13 | 18 | 16 | —^{d} | (13-34) |
| gp120 | 7 | 28 | 26 | — | (13-41) |
| V3 | 7 | 34 | 19 | — | (13-36) |
| MPER, CD4BS, CD4i | 0 | N/A | — | — | — |

^{a}N = number of plasma donor subjects that showed an elevation during the follow up period.
^{b}Median time from T₀, first day viral load reaches 100 copies/ml.,
^{c}ID = immunodominant.
^{d}Upper confidence limit is infinite (Elevations can occur greater than >67 days from T₀).

TABLE 2
Timing of Immune Complexes and free HIV Antibodies Relative to T₀.

| | Days | | | IgG IC | IgM IC | VL^{d} |
|---|---|---|---|---|---|---|
| | post | Anti-HIV- | Custom | (RNA | (RNA | (RNA |
| Subject | T₀ | Abbott^{a} | IgM^{b} | copies) | copies) | copies/ml) |
| 12008 | 2 | 0.181 | 0.017 | —^{c} | — | 925 |
| | 4 | 0.157 | 0.018 | — | — | 24,194 |
| | 9 | 0.173 | 0.014 | 32,833 | 25,389 | 5,631,550 |
| | 14 | 1.795 | 0.114 | 130,556 | 162,222 | 6,486,240 |
| | 16 | 9.528 | 0.795 | 132,778 | 89,056 | 2,296,060 |
| | 21 | 6.063 | 0.997 | 1,344 | 778 | 26,311 |
| | 23 | 4.646 | 0.872 | — | 2,350 | 17,425 |
| 9079 | 4 | 0.108 | 0.045 | — | — | 56,508 |
| | 6 | 0.135 | 0.037 | — | 989 | 731,600 |
| | 11 | 2.839 | 0.618 | 788 | 9,944 | 584,913 |
| | 13 | 10.763 | 1.186 | 190 | 652 | 2,170,000 |
| | 19 | 4.169 | 0.610 | — | — | 20,988 |
| | 26 | 6.331 | 0.353 | — | — | 3,594 |
| 9076 | −3 | 0.164 | 0.001 | — | — | <50 |
| | 3 | 0.155 | −0.016 | — | — | 5,393 |
| | 5 | nt | 0.039 | — | — | 352 |
| | 11 | 4.879 | 0.345 | — | — | 697,960 |
| | 14 | 17.241 | 0.958 | 3,256 | 1,489 | 507,740 |
| | 19 | 17.241 | 0.121 | — | — | 4,912 |
| 9015 | 0 | 0.171 | 0.030 | — | — | 59 |
| | 7 | 0.171 | 0.023 | 1,439 | 1,422 | 2,483,020 |
| | 9 | 0.171 | 0.036 | 1,933 | 2,067 | 2,912,250 |
| | 14 | 7.863 | 0.120 | — | 926 | 349,278 |
| | 16 | 11.538 | 0.255 | — | — | 276,354 |
| 9021 | −3 | 0.483 | 0.055 | — | — | <50 |
| | 1 | 0.793 | 0.069 | — | — | 387 |
| | 5 | 0.569 | 0.096 | — | 376 | 28,487 |
| | 8 | 0.138 | 0.085 | — | 4,922 | 404,218 |
| | 12 | 0.716 | 0.213 | — | 7,156 | 520,471 |
| | 15 | 2.345 | 1.251 | 1,314 | 29,833 | 5,417,090 |
| 9077 | 2 | 0.153 | 0.071 | — | — | 1353 |
| | 6 | 0.198 | 0.078 | — | — | 79,235 |
| | 11 | 0.135 | 0.091 | — | — | 798,590 |
| | 13 | 0.432 | 0.093 | 8,494 | 5,670 | 400,900 |
| | 18 | 8.793 | 1.682 | — | — | 1,114,040 |
| | 25 | 9.514 | 1.267 | — | — | 40,369 |
| | 28 | 10.225 | 0.861 | — | — | 17,838 |

^{a}ABBOTT Anti-HIV 1/2 EIA (Abbott Diagnostics), positivity cutoff >1.0.
^{b}In house IgM ELISA, O.D. antigen-O.D. no antigen, positivity criteria ≧0.1 and ≧3 fold baseline.
^{c}<200 copies/ml by quantitative real time PCR of immune complexes.
^{d}HIV-1 RNA PCR Ultra, Quest Diagnostics, Lyndhurst NJ.
Bold font indicates positive value.

TABLE 3
Comparison of the ability of the target-cell limited model and variants including anti-gp41
antibodies to fit early plasma VL kinetic data. The viral load data from each donor was fit
using the target cell-limited model and models that incorporated the effects of anti-gp41 IgG,
IgM and IgG + IgM.

| | | | CE | | | ID | | | CDE | Best |
|---|---|---|---|---|---|---|---|---|---|---|
| | ^{a}CE | CE, | IgG+ | ^{b}ID | ID | IgG+ | ^{c}CDE | CDE | IgG+ | Fitting |
| Subject | IgG | IgM | IgM | IgG | IgM | IgM | IgG | IgM | IgM | Model* |
| Pt. 6240 | 0.320 | 0.326 | 0.266 | 0.975 | 0.977 | 0.975 | 0.975 | 0.975 | 1.000 | Target-cell |
| | | | | | | | | | | limited |
| Pt. 6246 | 1.000 | 1.000 | 1.000 | 1.000 | 1.000 | 1.000 | 1.000 | 1.000 | 1.000 | Target-cell |
| | | | | | | | | | | limited |
| Pt. 9032 | 0.004 | 0.006 | 0.001 | 0.005 | 0.006 | 0.006 | 0.89 | 0.011 | 0.923 | CE or ID |
| Pt. 9077 | 0.567 | 0.998 | 0.687 | 0.604 | 0.998 | 0.623 | 0.722 | 0.998 | 0.991 | Target-cell |
| | | | | | | | | | | limited |
| Pt. 9079 | 0.992 | 0.994 | 0.960 | 0.998 | 0.649 | 0.992 | 0.964 | 0.070 | 0.598 | Target-cell |
| | | | | | | | | | | limited |
| Pt. 12008 | 0.566 | 0.607 | 0.590 | 0.648 | 0.546 | 0.657 | 1.000 | 1.000 | 1.000 | Target-cell |
| | | | | | | | | | | limited |

^{a}CE = Clearance Enhanced,
^{b}ID = Infectivity Diminished,
^{c}CDE = Cell Death Enhanced.
The models with antibody effects include one additional parameter, which when set to zero reduces the model to the target-cell limited model. An F-test was used to determine if any of the models including antibody fit the VL data significantly better than the target-cell model. The Table gives the p-values computed from the F-test. A p-value <0.05 indicates a model including antibody fit better than the target-cell limited model.

TABLE 4
Neutralizing Antibodies to MN Trinidad Cohort

| | Time | Peak Titer |
|---|---|---|
| PtID | (Months) | IC50 |
| SC03 | 10 | 812 |
| SC05 | 10 | 813 |
| SC11 | 3 | 1552 |
| SC24 | 6 | 1907 |
| SC25 | 13 | 361 |
| SC41 | 9 | 364 |
| SC42 | 2 | 8603 |
| SC44 | 4 | 609 |
| SC46 | 9 | 127 |
| SC51 | 6 | 385 |

What is claimed is:

1. A method of identifying a subject who is positive for HIV RNA but negative for anti-HIV antibodies comprising:
i) obtaining a biological sample from said subject,
ii) contacting said sample with anti-IgM; anti-IgG, anti-IgA or anti-C3D antibodies bound to a solid support under conditions such that antibody-HIV virion complexes present in said sample can bind to said antibodies,
iii) washing said solid support under conditions such that uncomplexed HIV virion is removed,
iv) treating said solid support under conditions such that viral RNA from antibody-HIV virion complexes bound to said solid support can be measured, and
v) assaying for viral RNA resulting from step (iv),
wherein the presence of said viral RNA indicates said subject is positive for HIV RNA but negative for anti-HIV antibodies.

2. The method according to claim 1 wherein said subject is a human.

3. The method according to claim 1 wherein said antibodies are anti-human IgM, IgG or IgA antibodies.

4. The method according to claim 1 wherein said biological sample is a plasma sample, a serum sample, a mucosal sample, a urine sample, a saliva sample or a rectal wash.

5. The method according to claim 4 wherein said mucosal sample is a seminal plasma sample or a cervical lavage sample.

6. The method according to claim 1 wherein said solid support is an ELISA plate.

7. The method according to claim 1 wherein, in step (ii), said sample is contacted with a protein G column.

8. The method according to claim 1 wherein, in step (iv), real-time reverse transcription PCR is used to assay for said viral RNA.

9. The method according to claim 1 wherein said method is a method of identifying a subject who is within 3 weeks of an HIV transmission event.

10. A method of detecting antibody-HIV virion complexes in a subject comprising:
i) obtaining a biological sample from said subject,
ii) contacting said sample with anti-IgM, anti-IgG, anti-IgA or anti-C3D antibodies bound to a solid support under conditions such that antibody-HIV virion complexes present in said sample can bind to said antibodies,
iii) washing said solid support under conditions such that uncomplexed HIV virion is removed,
iv) treating said solid support under conditions such that viral RNA from antibody-HIV virion complexes bound to said solid support can be measured, and
v) assaying for viral RNA resulting from step (iv), and thereby effecting said detection.

11. The method according to claim 10 wherein said subject is a human.

12. The method according to claim 10 wherein said antibodies are anti-human IgM, IgG or IGA antibodies.

13. The method according to claim 10 wherein said biological sample is a plasma sample, a serum sample, a mucosal sample, a urine sample, a saliva sample or a rectal wash.

14. The method according to claim 13 wherein said mucosal sample is a seminal plasma sample or a cervical lavage sample.

15. The method according to claim 10 wherein said solid support is an ELISA plate.

16. The method according to claim 10 wherein, in step (ii), said sample is contacted with a protein G column.

17. The method according to claim 10 wherein, in step (iv), real-time reverse transcription PCR is used to assay for said viral RNA.

18. A purified antibody having the variable heavy and variable light chains of C14-2 shown in FIG. 15F, or scFv, Fv, Fab′, Fab or F(ab′)₂ fragment thereof.

19. A composition comprising the antibody, or fragment thereof, of claim 18 and a carrier.

20. A purified antibody having the heavy chain of F3 shown in FIG. 15K, or scFv, Fv, Fab′, Fab or F(ab′)₂ fragment thereof.

21. A composition comprising the antibody, or fragment thereof, of claim 20 and a carrier.


SEQUENCE LISTING (8 sequences)

SEQ ID NO: 1 - protein, 44 aa, Artificial Sequence
  Description of Artificial Sequence Synthetic polypeptide
RVLAVERYLRDQQLLGIWGCSGKLICTTAVPWNASWSNKSLNKI

SEQ ID NO: 2 - protein, 20 aa, Artificial Sequence
  Description of Artificial Sequence Synthetic peptide
QQEKNEQELLELDKWASLWN

SEQ ID NO: 3 - protein, 16 aa, Artificial Sequence
  Description of Artificial Sequence Synthetic peptide
SLWNWFNITNWLWYIK

SEQ ID NO: 4 - protein, 33 aa, Artificial Sequence
  Description of Artificial Sequence Synthetic polypeptide
TRPNNNTRKSIHIGPGRAFYTTGEIIGDIRQAH

SEQ ID NO: 5 - protein, 33 aa, Artificial Sequence
  Description of Artificial Sequence Synthetic polypeptide
TRPNNNTRKSIRIGPGQAFYATGDIIGDIRQAH

SEQ ID NO: 6 - DNA, 255 bp, Artificial Sequence
  Description of Artificial Sequence Synthetic polynucleotide
gaggtkcagctggtggagtctgggggaggcttagttcagcctggggggtccctgagactctcctgtgcagcctctggattcaccttcagtagttactggatgcactgggtccgccaagctccagggaaggggctggtgtgggtctcacgtattaatagtgatgggagtagcacagactacgcggactccgtgaagggccgattcaccatctccagagacaacgccaagaacacgctatatctgcaaatgagcagt

SEQ ID NO: 7 - DNA, 255 bp, Artificial Sequence
  Description of Artificial Sequence Synthetic polynucleotide
cagtctgtgytgackcagccaccctcagcgtctgggacccccgggcagagggtcaccatctcttgttctggaagcagctccaacatcggaagtaatactgtaaactggtaccagcagctcccaggaacggcccccaaactcctcatctataggaataatcagcggccctcaggggtccctgaccgattctctggctccaagtctggcacctcagcctccctggccatcagtgggctccaggctgaggatgaggct

SEQ ID NO: 8 - DNA, 423 bp, Artificial Sequence
  Description of Artificial Sequence Synthetic polynucleotide
gaggtkcagctggtggagtctgggggaggcttggtacagcctggggggtccctgagactctcctgtgcagcctctggtttcacctttagcagctatgccatgacctgggtccgccaggctccagggaaggggctggagtgggtctcaggtattagtggtagtggtggtagcacaaacgacgcagactccgtgaagggtcggttcaccatctccagagacaattccaagaacacgctgtatctgcaaatgaacagtctgagagccgaggacacggccatatattactgtgcgaaaacgatttgtagtggaggtagttgcccctggggctacttcgatttctggggccgtggcaccctggtcactgtctcctcagggagtgcatccgccccaacccttttccccctcgtctcctgtgagaattcc